(19) United States
(12) Patent Application Publication
(10) Pub. No.: US 2026/0078121 A1
(43) Pub. Date: Mar. 19, 2026

(54) INHIBITORS OF ENL/AF9 YEATS AND FLT3

(71) Applicant: Bridge Medicines, New York, NY (US)

(72) Inventors: Tammy Ladduwahetty, London (GB); Joseph P. Vacca, Telford, PA (US); Sébastien L. Degorce, Hertfordshire (GB)

(21) Appl. No.: 19/109,792

(22) PCT Filed: Aug. 18, 2023
(86) PCT No.: PCT/US2023/072515

§ 371 Date: Mar. 7, 2025

Related U.S. Application Data
(60) Provisional application No. 63/404,659, filed on Sep. 8, 2022.

(51) Int. Cl. C07D 471/04; A61K 31/437; A61K 31/444; A61K 31/4545; A61K 45/06; A61P 35/02

(52) U.S. Cl. CPC C07D 471/04; A61K 31/437; A61K 31/444; A61K 31/4545; A61K 45/06; A61P 35/02

(57) ABSTRACT

Compounds and pharmaceutical compositions comprising compounds that inhibit ENL/AF9 YEATS and FLT3 are disclosed herein. Methods for suppressing oncogene expression in a cell, or for treating acute leukemias, using the compounds and pharmaceutical compositions comprising the compounds are also disclosed. The compounds, pharmaceutical compositions and methods can be used to inhibit key drivers of cancer and cancer stem cell survival.

### CROSS-REFERENCE TO RELATED APPLICATION

This application claims priority to U.S. Provisional Application No. 63/404,659, filed Sep. 8, 2022, which is incorporated herein in its entirety.

### FIELD OF THE INVENTION

The present application relates generally to compounds that inhibit ENL/AF9 YEATS and FLT3 and therapeutic methods of using such compounds. The compounds and methods find use in treating a variety of different diseases, including blood cancers such as leukemia.

### BACKGROUND OF THE INVENTION

The epigenome is an ensemble of chemical compounds contiguous to the DNA, responsible for the modification of the genome without altering the DNA sequences. It is dynamically regulated by chemical changes of DNA, RNA, and histones, around which DNA is packaged. It has been demonstrated that mutations in genes encoding epigenetic regulators plays a role in acute myeloid leukemia (AML) pathogenesis (Shih A H, Abdel-Wahab O, Patel J P, et al. “The role of mutations in epigenetic regulators in myeloid malignancies.” Nat. Rev. Cancer 2012; 12:599-612).

ENL is a chromatin reader protein possessing an amino-terminal YEATS domain (named for the first-discovered members of the family: Yaf9, ENL, AF9, Taf14, Sas5) and a disordered carboxy-terminal protein-protein interaction (PPI) interface. YEATS are a family of histone acetyllysine readers that act as effectors by allowing chromatin to be more accessible to RNA polymerase and transcriptional factors. Erb, et al. reported that a disproportionate number of leukemia proto-oncogenes and dependencies have ENL at their promoters (Erb, M. A. et al., “Transcription control by the ENL YEATS domain in acute leukaemia,” Nature 543, 270-274 (2017). Wan, et al. found that ENL binds to acetylated histone H3, and then colocalizes with H3K27 and H3K9ac on the promoters of genes essential for leukemia, and that ENL is required for AML maintenance (Wan L., et al. “ENL links histone acetylation to oncogenic gene expression in acute myeloid leukaemia,” Nature 2017; 543:265-9).

Given ENL's role in proliferation of leukemias, inhibitors of the YEATS domain of ENL are potential targets for treatment of blood cancers. For instance, Moustakim, et al. described small molecule inhibitors of ENL YEATS domain (Moustakim, M., et al., “Discovery of an MLLT1/3 YEATS Domain Chemical Probe,” Angew. Chem. Int. Ed. 2018, 57, 16302-16307). Moustakim's inhibitors compound contains a cyclic, nitrogenous heterocycle connected through a nitrogen atom to methylene group attached to a benzimidazole core.

FLT3 (Fms-like tyrosine kinase 3, FLK2) is a class III receptor tyrosine kinase. It is activated by the FLT3 ligand (FL) and signals through the PI3K, RAS, and JAK/STAT pathways (Scholl C. et al., Semin. Oncol., 35:336-45 (2008); Meshinchi S. et al., Clin. Cancer Res., 15:4263-9 (2009)). FLT3 plays a role in early hematopoiesis and FLT3 deficient mice have reduced numbers of progenitors of multiple lymphoid lineages (Mackarehtschian K, et al., Immunity, 3:147-61 (1995). Activating mutations in FLT3 are found in approximately 30% of AML patients, representing the most frequent genetic alteration in the disease. About 75% of the activating mutations are internal tandem duplications (ITD) and 25% are point mutations in the activation loop of the kinase domain. The most frequently identified activating point mutation is D835Y (Yamamoto et al., Blood, 97(8): 2434-2439 (2001)). However, mutations have also been found at N841I (Jiang, J. et al., Blood, 104(6): 1855-1858 (2004)) and Y842C (Kindler et al., Blood, 105(1): 335-340 (2005)). Additional point mutations have been identified in the juxtamembrane domain and kinase domain, although these have been shown to result in lower transforming potential (Reindel et al., Blood 107(9): 3700-3707 (2006)).

Murine bone marrow transplanted with a retrovirus expressing the FLT3-ITD has been shown to result in the production of a lethal myeloproliferative disease in mice (Kelly et al., Blood 99: 310-318 (2002)) characterized by leukocytosis consisting of mature neutrophils. This disease did not show a block in differentiation as seen in human AML suggesting that FLT3 mutations confer a proliferative or survival advantage to the cells.

A number of FLT3 inhibitors have been tested in clinical trials. Although they have shown initial clinical responses in AML, the responses observed were transient and resistance can develop rapidly (Weisberg, E. et al., Oncogene, 29:5120-34 (2010)). The major resistance mechanism appears to be through the acquisition of secondary mutations in FLT3, which may interfere with the binding of FLT3 inhibitors to the FLT3 receptor (Weisberg, E. et al., Oncogene, 29:5120-34 (2010); Chu, S. H. et al., Drug Resist. Update, 12:8-16 (2009)). Combinations of FLT3 inhibitors with chemotherapy are being tested in clinical trials despite the recognition that chemotherapy is poorly tolerated. Additional possible mechanisms for lack of durable responses include inadequate target coverage (Pratz, K. W., et al., Blood, 139:3938-46 (2009)) and protection of AML cells in the bone marrow where stromal growth factors may provide proliferative signals in addition to FLT3 activation (Tam, W. F. et al., Best Pract. Res. Clin. Haematol., 21:13-20 (2008)).

There remains a need for improved inhibitors useful for treating blood cancers.

### SUMMARY OF THE INVENTION

The invention is directed to compounds, pharmaceutical compositions, and methods for inhibiting YEATS/ENL and FLT3 and thereby treating various cancers, particularly blood cancers such as leukemia. The compounds, pharmaceutical compositions, and methods disclosed herein may be used to inhibit key drivers of cancer and cancer stem cell survival, thereby providing enhanced anti-cancer activity. In particular, the compounds, pharmaceutical compositions, and methods disclosed herein may be used to inhibit tyrosine kinase activity via FLT3 and the epigenetic driver ENL-YEATS. As such, cancer cells dependent on one or both of these pathways may be effectively treated with less dependency upon molecular diagnostics.

In a first aspect, compounds of Formula I are provided: which, in some embodiments, inhibit both YEATS/ENL and FLT3:

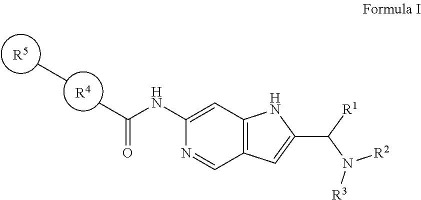

wherein:
R¹ and R² taken together form a pyrrolidine or piperidine;
R³ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

In a second aspect, compounds of Formula II are provided which, in some embodiments, inhibit both YEATS/ENL and FLT3:

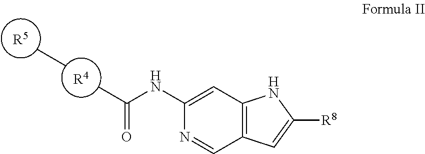

wherein:
R⁸ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

In a third aspect, the present invention relates to pharmaceutical composition comprising a compound of Formula I and/or Formula II and one or more pharmaceutically acceptable carriers. The pharmaceutical compositions can further comprise one or more therapeutic agents. Exemplary therapeutic agents include Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators and chemotherapeutic agents.

In a fourth aspect, the present invention relates to methods of treating acute leukemias comprising administering a therapeutically effective amount of a compound described herein or a pharmaceutical composition comprising the same to a subject in need thereof. The acute leukemia can be acute lymphoblastic leukemia (ALL) or acute myelogenous leukemia (AML). The method can further comprise administration of one or more additional therapeutic agents, e.g., Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators and chemotherapeutic agents. In a particular embodiment, one or more compounds of the present invention is administered with another FLT3 inhibitor, simultaneously or sequentially. In another particular embodiment, one or more compounds of the present invention is administered with a chemotherapeutic agent, simultaneously or sequentially. In certain embodiments, the chemotherapeutic agent is a drug for use in the treatment of AML, for example cytarabine, a BCL-2 inhibitor (e.g., venetoclax), or a menin inhibitor.

### DETAILED DESCRIPTION OF THE INVENTION

### I. Definitions

As used herein, “acyl” refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself is called “oxo”.

As used herein, the term “alkyl” includes linear or branched hydrocarbon structures. Lower alkyl refers to alkyl groups of from 1 to 6 carbon atoms. Examples of lower alkyl groups include methyl, ethyl, propyl, isopropyl, butyl, s- and t-butyl and the like. Preferred alkyl groups are those of C₂₀ or below, e.g., C₁-C₁₀ alkyl, C₁-C₈ alkyl and C₁-C₆ alkyl.

As used herein, “aryl” and “heteroaryl” mean (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzimidazole, quinoline, isoquinoline, quinoxaline, pyrimidine, pyrazine, tetrazole and pyrazole. As used herein aryl and heteroaryl refer to residues in which one or more rings are aromatic, but not all need be.

As used herein, “arylalkyl” refers to a substituent in which an aryl residue is attached to the parent structure through alkyl. Examples are benzyl, phenethyl and the like. “Heteroarylalkyl” refers to a substituent in which a heteroaryl residue is attached to the parent structure through alkyl. In one embodiment, the alkyl group of an arylalkyl or a heteroarylalkyl is an alkyl group of from 1 to 6 carbons. Examples include, e.g., pyridinylmethyl, pyrimidinylethyl and the like.

As used herein, “C₁ to C₂₀ hydrocarbon” or “C₁ to C₂₀ hydrocarbyl” (as a substituent) includes alkyl, cycloalkyl, polycycloalkyl, alkenyl, alkynyl, aryl and combinations thereof. Examples include cyclopropylmethyl, benzyl, phenethyl, cyclohexylmethyl, camphoryl and naphthylethyl. Hydrocarbon refers to any substituent comprised of hydrogen and carbon as the only elemental constituents. Cycloalkyl is a subset of hydrocarbyl and includes cyclic hydrocarbon groups of from 3 to 8 carbon atoms. Examples of cycloalkyl groups include c-propyl, c-butyl, c-pentyl, norbornyl and the like.

“Alkoxy” or “alkoxyl” refers to groups of from 1 to 8 carbon atoms of a straight, branched or cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. For the purpose of this application, alkoxy and lower alkoxy include methylenedioxy and ethylenedioxy.

As used herein, “carbocycle” is includes ring systems in which the ring atoms are all carbon but of any oxidation state. Thus (C₃-C₈) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

As used herein, the term “therapeutically effective amount” refers to any amount of a compound of the present invention or any other pharmaceutically active agent which, as compared to a corresponding a patient who has not received such an amount of the compound of the present invention or the other pharmaceutically active agent, results in improved treatment, healing, prevention, or amelioration of a disease, disorder, or side effect, or a decrease in the rate of advancement of a disease or disorder.

As used herein, the term “fused bicycles” refers to bicyclic carbocycles and bicyclic heterocycles in which each ring (a carbocycle or heterocycle) shares two adjacent atoms with another ring (a carbocycle or heterocycle). Each ring of the fused carbocycle can be selected from non-aromatic or aromatic rings. In preferred embodiments, the aromatic ring, such as phenyl, may be fused to another aromatic ring. In other embodiments, the aromatic ring may be fused to a non-aromatic ring, for example, cyclohexane, cyclopentane, or cyclohexene. Exemplary fused bicycles include 6,6; 6,5; and 5,6 fused bicyclic systems, wherein each number indicates the number of atoms in each ring. The fused bicycle can be substituted at any one or more position where it can have a hydrogen atom. The fused bicycle is bonded to the parent structure at the first numbered ring, e.g., the “6” ring of a fused 6,5 bicycle.

As used herein, “heterocycle” means a cycloalkyl or aryl carbocycle residue in which from one to four carbons is replaced by a heteroatom selected from the group consisting of N, O and S. The nitrogen and sulfur heteroatoms may optionally be oxidized, and the nitrogen heteroatom may optionally be quaternized. Unless otherwise specified, a heterocycle may be non-aromatic or aromatic. Examples of heterocycles that fall within the scope of the invention include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. It is to be noted that heteroaryl is a subset of heterocycle in which the heterocycle is aromatic. Non-limiting examples of heteroaromatic rings include furan, benzofuran, isobenzofuran, pyrrole, indole, isoindole, thiophene, benzothiophene, imidazole, benzimidazole, purine, pyrazole, indazole, oxazole, benzoxazole, isoxazole, benzisoxazole, thiazole, benzothiazole, triazole, tetrazole, pyridine, quinoline, isoquinoline, pyrazine, quinoxaline, acridine, pyrimidine, quinazoline, pyridazine, cinnoline, phthalazine, and triazine. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

An oxygen heterocycle is a heterocycle containing at least one oxygen in the ring; it may contain additional oxygen atoms, as well as other heteroatoms. A sulfur heterocycle is a heterocycle containing at least one sulfur in the ring; it may contain additional sulfur atoms, as well as other heteroatoms. Oxygen heteroaryl is a subset of oxygen heterocycle; non-limiting examples include furan and oxazole. Sulfur heteroaryl is a subset of sulfur heterocycle; examples include thiophene and thiazine. A nitrogen heterocycle is a heterocycle containing at least one nitrogen in the ring; it may contain additional nitrogen atoms, as well as other heteroatoms. Non-limiting examples include piperidine, piperazine, morpholine, pyrrolidine and thiomorpholine. Nitrogen heteroaryl is a subset of nitrogen heterocycle; non-limiting examples include pyridine, pyrrole and thiazole.

As used herein, the term “optionally substituted” may be used interchangeably with “unsubstituted or substituted.” The term “substituted” refers to the replacement of one or more hydrogen atoms in a specified group with a specified radical. For example, substituted aryl, heterocyclyl etc. refer to aryl or heterocyclyl wherein one or more hydrogen atoms in each residue are replaced with halogen, haloalkyl, alkyl, acyl, alkoxyalkyl, hydroxyloweralkyl, carbonyl, phenyl, heteroaryl, benzenesulfonyl, hydroxy, loweralkoxy, haloalkoxy, oxaalkyl, carboxy, alkoxycarbonyl [—C(═O)O-alkyl], carboxamido [—C(═O)NH₂], alkylaminocarbonyl [—C(═O)NH-alkyl], cyano, acetoxy, nitro, amino, alkylamino, dialkylamino, dialkylaminoalkyl, dialkylaminoalkoxy, heterocyclylalkoxy, arylalkyl, (cycloalkyl)alkyl, heterocyclyl, heterocyclylalkyl, alkylaminoalkyl, heterocyclylaminoalkyl, heterocyclylalkylaminoalkyl, cycloalkylaminoalkyl, cycloalkylalkylaminoalkyl, arylaminoalkyl, and arylalkylaminoalkyl, mercapto, alkylthio, alkylsulfinyl, benzyl, heterocyclyl, phenoxy, benzyloxy, heteroaryloxy, aminosulfonyl, amidino, guanidino, and ureido. (C₁₋₆)hydrocarbyl, —SO₂alkyl, —SO₂NH₂, or —SO₂NHalkyl.

As used herein, “oxaalkyl” refers to alkyl residues in which one or more carbons (and their associated hydrogens) have been replaced by oxygen. Examples include methoxypropoxy, 3,6,9-trioxadecyl and the like. Alkoxy is a subset of oxaalkyl in which the carbon at the point of attachment is replaced by oxygen. The term oxaalkyl is intended as it is understood in the art [see Naming and Indexing of Chemical Substances for Chemical Abstracts, published by the American Chemical Society, 196, but without the restriction of 127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Non-limiting examples include ethylaminoethyl and methylthiopropyl.

As used herein, “solvate” refers to a compound in the solid state, wherein molecules of a suitable solvent are incorporated in the crystal lattice along with the compound. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

As used herein, the term “subject” or “subject in need thereof” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

### II. Compounds

In one aspect, compounds of Formula I are provided:

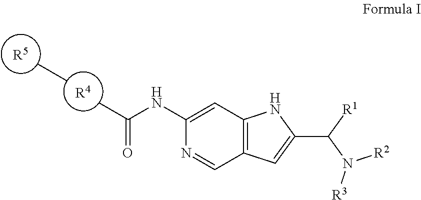

wherein:
R¹ and R² taken together form a pyrrolidine or piperidine;
R³ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

Exemplary R⁴ groups include, but are not limited to, benzene, pyridine, pyrimidine, pyridazine and pyrazine.

Exemplary R⁵ groups include, but are not limited to, pyrrolidine; pyrroline; pyrazolidine; pyrazoline; imidazoline; imidazoline; pyrrole; pyrazole; imidazole; triazole; isoxazole; oxazole; 1,2,3-oxadiazole; 1,3,4-oxadiozole; furazan; 1,2,4-oxadiazole; 1,2,3,4-oxatriazole; 1,2,3,5-oxatriazole; isothiazole; thiazole; 1,2,3-thiadiazole; 1,3,4-thiadizaole; 1,2,5-thadiazole; 1,2,4-thiadiazole; 1,2,3,4-thiatriazole; 1,2,3,5-thiatriazole; furan and thiophene.

In certain embodiments wherein R⁴ is a heterocycle, the heteroatom of R⁴ does not directly bond with the neighboring amide carbon or R⁵. Similarly, in certain embodiments wherein R⁵ is a heterocycle, the heteroatom of R⁵ does not directly bond with R⁴.

In a more particular embodiment, compounds of Formula Ia′ are provided:

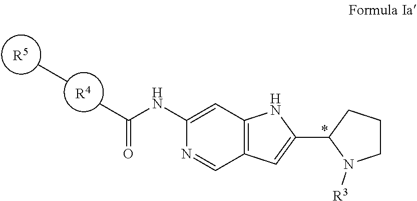

wherein R³, R⁴ and R⁵ are as defined above for Formula I.

In one embodiment, R³ is methyl. In another embodiment, R⁴ is benzene, optionally substituted as described above. In still another embodiment, R⁴ is pyrimidine, optionally substituted as described above. In a yet another embodiment, R⁵ is pyrazole, optionally substituted as described above. In a more particular embodiment, R³ is methyl; R⁴ is benzene or pyrimidine, optionally substituted as described above; and R⁵ is pyrazole, optionally substituted as described above. The compound can have the R- or S-configuration at the chiral center (*).

In another more particular embodiment, compounds of Formula Ia″ are provided:

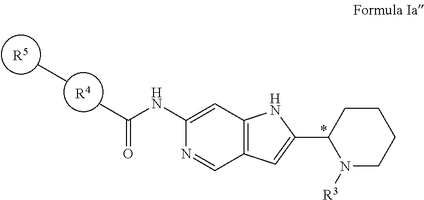

wherein R³, R⁴ and R⁵ are as defined above for Formula I.

In one embodiment, R³ is methyl. In another embodiment, R⁴ is benzene, optionally substituted as described above. In still another embodiment, R⁴ is pyrimidine, optionally substituted as described above. In a yet another embodiment, R⁵ is pyrazole, optionally substituted as described above. In a more particular embodiment, R³ is methyl; R⁴ is benzene or pyrimidine, optionally substituted as described above; and R⁵ is pyrazole, optionally substituted as described above. The compound can have the R- or S-configuration at the chiral center (*).

In another embodiment, compounds of Formula Ib are provided:

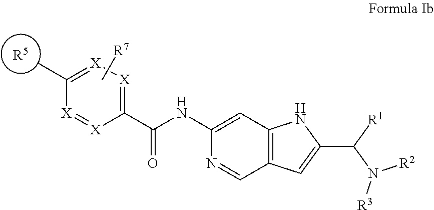

wherein R¹, R², R³, R⁵, and R⁷ are as defined above for Formula I; and
each X is independently selected from CH and N.

In a more particular embodiment, compounds of Formula Ib′ are provided:

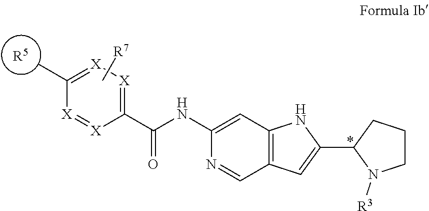

wherein R³, R⁵, and R⁷ are as defined above for Formula I; and
each X is independently selected from CH and N.

In a particular embodiment, R³ is methyl. In another particular embodiment, at least one X is N. In still another particular embodiment, all X are C. In yet another particular embodiment, R⁵ is pyrazole, optionally substituted as described above. In a more particular embodiment, R³ is methyl, at least one X is N, and R⁵ is pyrazole, optionally substituted as described above. In another more particular embodiment, R³ is methyl, all X are C, and R⁵ is pyrazole, optionally substituted as described above. The compound can be in the R- or S-configuration at the chiral center (*).

In another more particular embodiment, compounds of Formula Ib″ are provided:

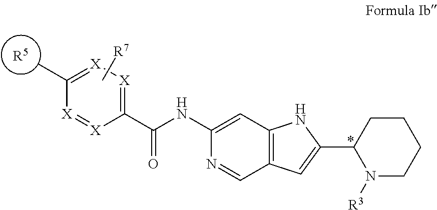

wherein R³, R⁵, and R⁷ are as defined above for Formula I; and
each X is independently selected from CH and N.

In a particular embodiment, R³ is methyl. In another particular embodiment, at least one X is N. In still another particular embodiment, all X are C. In yet another particular embodiment, R⁵ is pyrazole, optionally substituted as described above. In a more particular embodiment, R³ is methyl, at least one X is N, and R⁵ is pyrazole, optionally substituted as described above. In another more particular embodiment, R³ is methyl, all X are C, and R⁵ is pyrazole, optionally substituted as described above. The compound can be in the R- or S-configuration at the chiral center (*).

In another more particular embodiment, compounds of Formula Ic are provided:

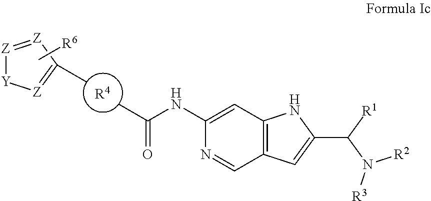

wherein R¹, R², R³, R⁴, and R⁶ are as defined above for Formula I;
each Z is independently selected from CH and N; and
Y is selected from NH and CH₂.

In a more particular embodiment, compounds of Formula Ic′ are provided:

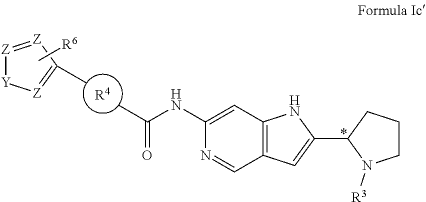

wherein R³, R⁴, and R⁶ are as defined above for Formula I;
each Z is independently selected from CH and N; and
Y is selected from NH and CH₂.

In a particular embodiment, R³ is methyl. In another particular embodiment, at least one Z is N and Y is NH. In another particular embodiment, R⁴ is benzene or pyrimidine, optionally substituted as described above. In a more particular embodiment, R³ is methyl, at least one Z is N, Y is NH, and R⁴ is benzene or pyrimidine, optionally substituted as described above. The compound can be in the R- or S-configuration at the chiral center (*).

In another more particular embodiment, compounds of Formula Ic″ are provided:

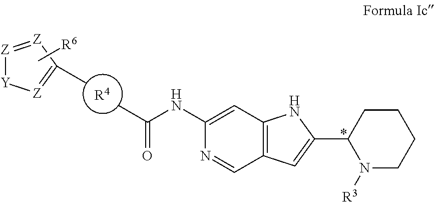

wherein R³, R⁴, and R⁶ are as defined above for Formula I;
each Z is independently selected from CH and N; and
Y is selected from NH and CH₂.

In a particular embodiment, R³ is methyl. In another particular embodiment, at least one Z is N and Y is NH. In another particular embodiment, R⁴ is benzene or pyrimidine, optionally substituted as described above. In a more particular embodiment, R³ is methyl, at least one X is N, Y is NH, and R⁴ is benzene or pyrimidine, optionally substituted as described above. The compound can be in the R- or S-configuration at the chiral center (*).

In another embodiment, compounds of Formula Id are provided:

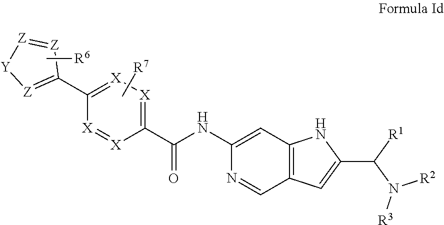

wherein R¹, R², R³, R⁶ and R⁷ are as defined above for Formula I;
each X is independently selected from CH and N;
each Z is independently selected from CH and N; and
Y is selected from CH₂ and NH.

In another particular embodiment, compounds of Formula Id′ are provided:

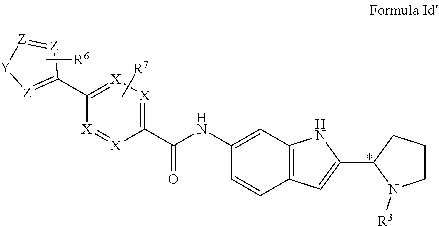

wherein R³, R⁶ and R⁷ are as defined above for Formula I;
each X is independently selected from CH and N;
each Z is independently selected from CH and N; and
Y is selected from CH₂ and NH.

In a particular embodiment, R³ is methyl. In another particular embodiment, at least one Z is N and Y is NH. In another particular embodiment, R³ is methyl, at least one X is N, at least one Z is N, and Y is NH. In another particular embodiment, R³ is methyl, all X are C, at least one Z is N, and Y is NH. The compound can be in the R- or S-configuration at the chiral center (*).

In another particular embodiment, compounds of Formula Id″ are provided:

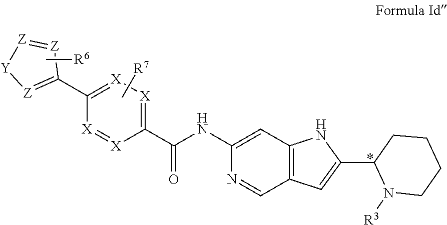

wherein R³, R⁶ and R⁷ are as defined above for Formula I;
each X is independently selected from CH and N;
each Z is independently selected from CH and N; and
Y is selected from CH and NH.

In a particular embodiment, R³ is methyl. In another particular embodiment, at least one Z is N and Y is NH. In another particular embodiment, R³ is methyl, at least one X is N, at least one Z is N, and Y is NH. In another particular embodiment, R³ is methyl, all X are C, at least one Z is N, and Y is NH. The compound can be in the R- or S-configuration at the chiral center (*).

As discussed, above, compounds described herein have a chiral center (*) and can be in the R or S-configuration. In one embodiment, the compound has the R-configuration, e.g., Formula Ia′ is:

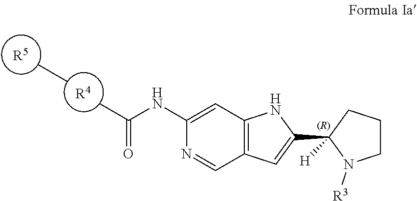

In another embodiment, the compound has the S-configuration, e.g., Formula Ia″ is:

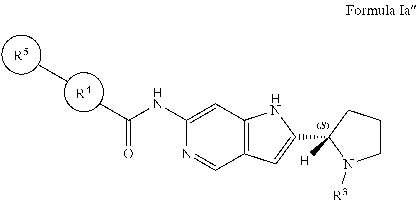

In another aspect, compounds of Formula II are provided:

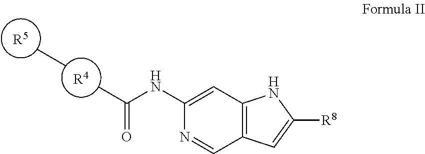

wherein:
R⁸ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

Exemplary R⁴ groups include, but are not limited to, benzene, pyridine, pyrimidine, pyridazine and pyrazine.

Exemplary R⁵ groups include, but are not limited to, pyrrolidine; pyrroline; pyrazolidine; pyrazoline; imidazoline; imidazoline; pyrrole; pyrazole; imidazole; triazole; isoxazole; oxazole; 1,2,3-oxadiazole; 1,3,4-oxadiozole; furazan; 1,2,4-oxadiazole; 1,2,3,4-oxatriazole; 1,2,3,5-oxatriazole; isothiazole; thiazole; 1,2,3-thiadiazole; 1,3,4-thiadizaole; 1,2,5-thadiazole; 1,2,4-thiadiazole; 1,2,3,4-thiatriazole; 1,2,3,5-thiatriazole; furan and thiophene.

In certain embodiments wherein R⁴ is a heterocycle, the heteroatom of R⁴ does not directly bond with the neighboring amide carbon or R⁵. Similarly, in certain embodiments wherein R⁵ is a heterocycle, the heteroatom of R⁵ does not directly bond with R⁴.

In one embodiment, compounds of Formula IIa are provided:

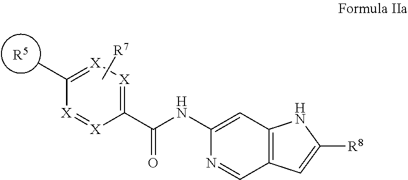

wherein R⁵, R⁷, and R⁸ are as defined above for Formula II; and
each X is independently selected from CH and N.

In one embodiment, R⁸ is methyl. In another embodiment, at least one X is N. In still another embodiment, all X are C. In yet another embodiment, R⁵ is pyrazole, optionally substituted as described above. In a more particular embodiment, R⁸ is methyl, at least one X is N and R⁵ is pyrazole, optionally substituted as described above. In another more particular embodiment, R⁸ is methyl, all X are C and R⁵ is pyrazole, optionally substituted as described above.

In another particular embodiment, compounds of Formula IIb are provided:

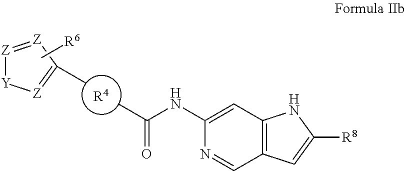

wherein R⁴, R⁶, and R⁸ are as defined above for Formula II;
each Z is independently selected from CH and N; and
Y is selected from NH and CH₂.

In one embodiment, R⁸ is methyl. In another embodiment, at least one Z is N. In still another embodiment, R⁴ is benzene or pyrimidine, optionally substituted as described above. In a particular embodiment, R⁸ is methyl, at least one Z is N, Y is NH, and R⁴ is benzene or pyrimidine, optionally substituted as described above.

In another embodiment, compounds of Formula IIc are provided:

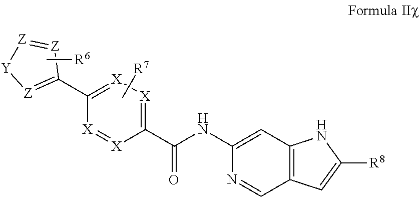

wherein R⁶, R⁷, and R⁸ are as defined above for Formula II;
each X is independently selected from CH and N;
each Z is independently selected from CH and N; and
Y is selected from CH₂ and NH.

In one embodiment, R⁸ is methyl. In another embodiment, at least one Z is N and Y is NH. In a particular embodiment, R⁸ is methyl, at least one X is N, at least one Z is N, and Y is NH. In another particular embodiment, R⁸ is methyl, all X are C, at least one Z is N, and Y is NH. The compound can be in the R- or S-configuration at the chiral center (*).

In another aspect, a compound of the present invention is selected from the group consisting of:

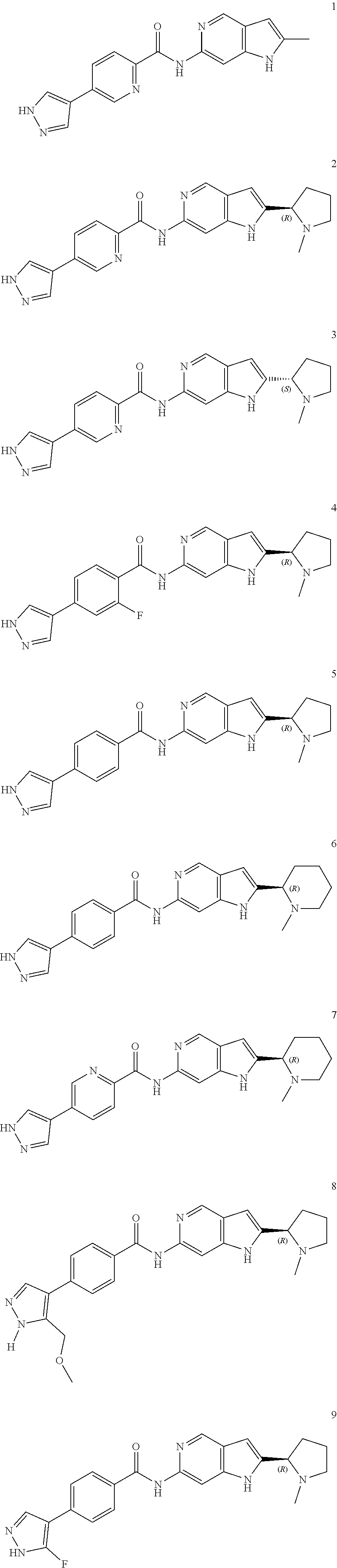

As used herein, “a compound”—unless expressly further limited—is intended to include salts of that compound. Thus, for example, the recitation “a compound of Formula I” as depicted above, would include salts:

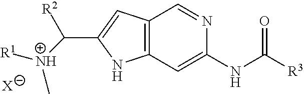

in which X is any counterion. In a particular embodiment, the term “compound of Formula I” refers to the compound or a pharmaceutically acceptable salt thereof. The term “pharmaceutically acceptable salt” refers to salts prepared from pharmaceutically acceptable non-toxic acids or bases including inorganic acids and bases and organic acids and bases. When the compounds of the present invention are basic, as they usually would be, salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. Further pharmaceutically acceptable salts include, when appropriate, nontoxic ammonium cations and carboxylate, sulfonate and phosphonate anions attached to alkyl having from 1 to 20 carbon atoms.

Compounds having R stereochemistry generally show higher activity than the corresponding S enantiomer. In other embodiments, the compound has a S stereochemical configurations at the chiral center.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### III. Pharmaceutical Compositions

The present invention also provides pharmaceutical compositions comprising at least one compound described herein (including pharmaceutically acceptable salts and solvates thereof).

A pharmaceutical composition comprises at least one compound described herein and one or more pharmaceutically acceptable excipients. Exemplary excipients include, but are not limited to, including, but not limited to, one or more binders, bulking agents, buffers, stabilizing agents, surfactants, wetting agents, lubricating agents, diluents, disintegrants, viscosity enhancing or reducing agents, emulsifiers, suspending agents, preservatives, antioxidants, opaquing agents, glidants, processing aids, colorants, sweeteners, taste-masking agents, perfuming agents, flavoring agents, diluents, polishing agents, polymer matrix systems, plasticizers and other known additives to provide an elegant presentation of the drug or aid in the manufacturing of a medicament or pharmaceutical product comprising a composition of the present inventions. Examples of carriers and excipients well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams &Wilkins, 2000; and Rowe, Raymond C. Handbook of Pharmaceutical Excipients. Chicago, Pharmaceutical Press, 2005.

Non-limiting examples of excipients include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), hydroxypropyl cellulose, titanium dioxide, talc, calcium carbonate (e.g., granules or powder), microcrystalline cellulose, powdered cellulose, dextrates, kaolin, silicic acid, sorbitol, starch, pre-gelatinized starch, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, a syloid silica gel (AEROSIL200, manufactured by W.R. Grace Co. of Baltimore, MD), a coagulated aerosol of synthetic silica (marketed by Degussa Co. of Plano, TX), CAB-O-SIL (a pyrogenic silicon dioxide product sold by Cabot Co. of Boston, MA), colorants and mixtures thereof.

The pharmaceutical compositions can optionally include one or more additional therapeutic agents.

Additional therapeutic agents include Bcl-2 inhibitors, cyclin-dependent kinase 4 and 6 (CDK 4/6) inhibitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators, and chemotherapeutic agents.

The standard of care for AML and ALL is currently chemotherapy with a chemotherapeutic agent. Exemplary chemotherapeutic agents include, but are not limited to, daunorubicin, cytarabine, methotrexate, mitoxantrone, methotrexate, mafosamide and vincristine.

Targeted therapeutic agents e.g., those discussed below, can be used alone or in combination with a chemotherapeutic agent.

Exemplary Bcl-2 inhibitors include, but are not limited to, e.g. oblimersen, navitoclax and venetoclax.

Exemplary cyclin-dependent kinases 4 and 6 (CDK 4/6) inhibitors include, but are not limited to, palbociclib, ribociclib and abemaciclib.

Epigenetic modulators include, but are not limited to, menin-histone methyltransferase MLL (i.e., menin-MLL) inhibitors, FLT3 inhibitors, P-TEFb inhibitors, histone methyltransferase inhibitors (e.g., DOT1L and EZH2 inhibitors), bromodomain and extra-terminal domain (BET) inhibitors and dihydroorotate dehydrogenase (DHODH) inhibitors.

Exemplary FLT3 inhibitors include, but are not limited to, sorafenib, lestaurtinib, sunitinib, tandutinib, quizartinib, midostaurin, gilteritinib, crenolanib, cabozantinib and ponatinib.

Combinations of epigenetic modulators, e.g., menin-MLL inhibitors and FLT3 inhibitors, are also contemplated as these have shown enhanced apotosis induction in AML models.

In one embodiment, the additional therapeutic agents comprise a combination of at least one Bcl-2 inhibitor and at least one FLT3 inhibitor.

Exemplary DNA methyltransferase inhibitors include, but are not limited to, azacytidine and decitabine.

Exemplary HDAC inhibitors include, but are not limited to, panobinostat and vorinostat.

Exemplary mTOR inhibitors include, but are not limited to, everolimus.

Exemplary glucocorticoids include, but are not limited to, dexamethasone and prednisolone.

Exemplary mutant isocitrate dehydrogenase inhibitors include, but are not limited to, ivosidenib (IDH1) and enasidenib (IDH2).

In one embodiment, the additional therapeutic agents comprise a combination of at least one isocitrate dehydrogenase inhibitor and at least one CDK 4/6 inhibitor.

### IV. Methods of Use

The present invention also relates to methods of using at least one compound described herein or a pharmaceutical composition described herein to suppress oncogene expression in a cell. In one embodiment, a method of suppressing oncogene expression in a cell comprises exposing the cell to at least one compound described herein. The present invention also relates to methods of using at least one compound described herein or a pharmaceutical composition described herein to treat an acute leukemia. In one embodiment, a method of treating an acute leukemia comprises administering a therapeutically effective amount of at least one compound described herein to a subject in need thereof.

Acute leukemias are rapidly progressing leukemia characterized by replacement of normal bone marrow by blast cells of a clone arising from malignant transformation of a hematopoietic cell. The acute leukemias include acute lymphoblastic leukemia (ALL) and acute myelogenous leukemia (AML). ALL often involves the CNS, whereas acute monoblastic leukemia involves the gums, and AML involves localized collections in any site (granulocytic sarcomas or chloromas).

In one embodiment, the acute leukemia is ALL. ALL is the most common malignancy in children, with a peak incidence from ages 3 to 5 years. It also occurs in adolescents and has a second, lower peak in adults. Typical treatment emphasizes early introduction of an intensive multidrug regimen, which may include prednisone, vincristine, anthracycline or asparaginase. Other drugs and combinations are cytarabine and etoposide, and cyclophosphamide. Relapse usually occurs in the bone marrow but may also occur in the CNS or testes, alone or concurrent with bone marrow. Although second remissions can be induced in many children, subsequent remissions tend to be brief.

In another embodiment, the acute leukemia is AML. The incidence of AML increases with age; it is the more common acute leukemia in adults. AML may be associated with chemotherapy or irradiation (secondary AML). Remission induction rates are lower than with ALL, and long-term disease-free survival reportedly occurs in only 20 to 40% of patients. Treatment differs most from ALL in that AML responds to fewer drugs. The basic induction regimen includes cytarabine; along with daunorubicin or idarubicin. Some regimens include 6-thioguanine, etoposide, vincristine, and prednisone. Clinical aspects of AML are reviewed by C. A. Schiffer and R. M. Stone in Cancer Medicine, Ed. David W. Kufe et al, 6th Edition, B.C. Decker, 2003.

This French, American, and British (FAB) classification has been developed to diagnose and classify acute myeloid leukemia. The diagnosis of acute myeloid leukemia requires that myeloblasts constitute 30% (or 20% based on a recent World Health Organization (WHO) classification system) or more of bone marrow cells or circulating white blood cells. The hematologic properties of the disease define the various subtypes described below. The FAB nomenclature (M1 through M7) classifies the subtypes of acute myeloid leukemia according to the normal marrow elements that the blasts most closely resemble. The following list includes both the FAB classifications as well as additional classes recognized by the WHO.
Acute myeloid leukemia, minimally differentiated (MO)
Acute myeloid leukemia without maturation (M1)
Acute myeloid leukemia with maturation (M2)
Acute myeloid leukemia with maturation with t(8;21)
Acute promyelocytic leukemia (M3)
Hypergranular type
Microgranular type
Acute myelomonocytic leukemia (M4)
Acute myelomonocytic leukemia with increased marrow eosinophils (M4E0)
Acute Monocytic Leukemia (M5)
Acute monoblastic leukemia (M5a)
Acute monocytic leukemia with maturation (M5b)
Erythroleukemia Erythroid/myeloid) (M6a)
Pure erythroid malignancy (M6b)
Acute megakaryoblastic leukemia (M7)
Acute megakaryoblastic leukemia associated with t(1;22)
Acute basophilic leukemia
Acute myelofibrosis (acute myelodysplasia with myelofibrosis)
Acute leukemia and transient myeloproliferative disorder in Down's Syndrome
Hypocellular acute myeloid leukemia
Myeloid sarcoma

In one embodiment, a method of treating a subtype of AML listed above comprises administering a therapeutically effective amount of at least one compound described herein to a subject in need thereof.

The at least one compound used in the present methods can be provided in the form of a pharmaceutical composition described hereinabove.

Routes of administration include enteral, such as oral; and parenteral, such as intravenous, intra-arterial, intramuscular, intranasal, rectal, intraperitoneal, subcutaneous and topical routes.

For parenteral administration, the active compounds may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water-soluble salt of the active agents. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or nonaqueous solution, dispersion, suspension or emulsion.

For oral administration, the active compounds may be combined with one or more solid inactive ingredients for the preparation of tablets, capsules, pills, powders, granules or other suitable oral dosage forms. For example, the active compounds may be combined with at least one excipient such as fillers, binders, humectants, disintegrating agents, solution retarders, absorption accelerators, wetting agents, absorbents or lubricating agents.

The specific doses of the active compound(s) employed in the composition and methods of the invention to obtain therapeutic benefit will, of course, be determined by the particular circumstances of the individual patient. Such circumstances include the size, weight, age and sex of the patient, the nature and stage of the disease, the aggressiveness of the disease, and the route of administration.

For the compounds described herein, the preferred daily dose is in the range of about 1 to about 10,000 mg, more preferably from about 5 to about 5,000 mg, still more preferably about 10 to about 3,000, most preferably about 50 to about 1,000, for example. In certain embodiments, the preferred daily dose is in the range of about 50 mg to about 4,000 mg, about 100 mg to about 3,000 mg, about 500 to about 2,000 or about 750 mg to about 1,500 mg. In other embodiments, the preferred daily dose is in the range of 2,000 mg to about 10,000 mg, about 3,000 to about 9,000 mg, about 4,000 mg to about 8,000 mg, or about 4,500 to about 7,500 mg.

A dose may be administered one to four times a day, e.g., once a day, as required to provide therapeutic benefit. In certain embodiments, a therapeutic compound of the invention is administered intravenously, either as a one-time dose or as part of a scheduled dosing regimen that may be spread out over several days, weeks, or months. The compounds of the invention may also be administered by periodic injection, as needed to obtain a therapeutic benefit.

The methods described herein can further comprise administration of an additional therapeutic agent, e.g., Bcl-2 inhibitors, cyclin-dependent kinase 4 and 6 (CDK 4/6) inhbitors, DNA methyltransferase inhibitors, histone deacetylase (HDAC) inhibitors, histone demethylase inhibitors, mTOR inhibitors, mutant isocitrate dehydrogenase (IDH1 and IDH2) inhibitors, glucocorticoids, epigenetic modulators, and chemotherapeutic agents. The additional therapeutic agent can be administered either simultaneously or sequentially with the compounds described herein. In some embodiments administration of a compound described herein and additional therapeutic agent can produce a synergistic effect.

### Examples

The following compounds have been prepared, isolated and characterized using the methods disclosed herein. They demonstrate a partial scope of the invention and are not meant to be limiting of the scope of the invention.

The compounds of the present invention were prepared by methods well known in the art of synthetic organic chemistry. During synthetic sequences it was sometimes necessary or desirable to protect sensitive or reactive groups on any of the molecules concerned. This was achieved by means of conventional protecting groups, such as those described in T. W. Greene and P. G. M. Wuts Greene's Protective Groups in Organic Synthesis, Fourth edition, John Wiley and Sons, 2006. The protecting groups were removed at a convenient subsequent stage using methods well known in the art.

All reactions were performed under a dry atmosphere of nitrogen unless otherwise specified. Indicated reaction temperatures refer to the reaction bath, while room temperature (rt) is noted as 25° C. Commercial grade reagents and anhydrous solvents were used as received from vendors and no attempts were made to purify or dry these components further. Removal of solvents under reduced pressure was accomplished with a Buchi rotary evaporator at approximately 28 mm Hg pressure using a Teflon-linked KNf vacuum pump. Flash column chromatography was carried out using a Teledyne Isco CombiFlash Companion unit with RediSep Rf silica gel columns. Proton NMR spectra were obtained on a 300 MHz and 400 MHz Bruker Nuclear Magnetic Resonance Spectrometer. Chemical shifts (δ) are reported in parts per million (ppm) and coupling constants (J) values are given in Hz, with the following spectral pattern designations: s, singlet; d, doublet; t, triplet; q, quartet; dd, doublet of doublet; m, multiplet; brs, broad singlet. Tetramethylsilane was used as an internal reference. Mass spectroscopic analysis were performed using positive and negative mode electron spray ionization (ESI) on an Agilent 1200 system. High pressure liquid chromatography (HPLC) purity analysis was performed using a Varian Pro Star HPLC system with a binary solvent system A and B using a gradient elution [A, H₂O with 0.0284% NH₄OAc and 0.0116% acetic acid; B, CH₃CN] and flow rate=1 mL/min, with PDA Scan for UV detection.

Abbreviations

| | |
|---|---|
| BINAP | 2,2′-Bis(diphenylphosphino)-1,1′-binaphthyl |
| CH₃CN | Acetonitrile |
| Cs₂CO₃ | Cesium Carbonate |
| DCM | Dichloromethane |
| DIPEA | Diisopropylethylamine |
| DMF | Dimethylformamide |
| DMP | Dess-Martin periodinane |
| EDCI | 1-Ethyl-3-(3-dimethylaminopropyl)carbodiimide |
| EA | Ethylactetate |
| EtOH | Ethyl alcohol |
| HCl | hydrochloric acid |
| K₂CO₃ | Potassium Carbonate |
| K₃PO₄ | Potassium Phosphate |
| LiHMDS | Lithium bis(trimethylsilyl)amide |
| MsCl | Methanesulfonyl chloride |
| MeOH | Methanol |
| NaHCO₃ | Sodium bicarbonate |
| NaOAc | Sodium acetate |
| NH₄Cl | Ammonium chloride |
| Pd(dppf)Cl₂•CH₂Cl₂ | -Bis(diphenylphosphino)ferrocene]dichloro- |
| | palladium(II), complex with dichloromethane |
| Pd(dppf)Cl₂ | - Bis(diphenylphosphino)ferrocene]dichloro- |
| | palladium(II) |
| PE | Petroleum ether |
| RT | room temperature |
| SEMCl | 2-(Trimethylsilyl)ethoxymethyl chloride |
| TFA | Trifluoroacetic acid |
| THF | Tetrahydrofuran |
| THP | Tetrahydropyran |
| TEA | Triethylamine |

### Intermediate 1: (R)-2-(1-methylpyrrolidin-2-yl)-1-((2-(trimethylsilyl)ethoxy)methyl)-1H-pyrrolo[3,2-c]pyridin-6-amine

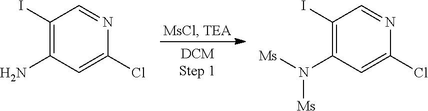 [SMILES: Nc1cc(Cl)ncc1I ; "MsCl, TEA" ; CS(=O)(=O)N(c1cc(Cl)ncc1I)S(C)(=O)=O]

Into a 20-L 4-necked round-bottom flask purged and maintained with an inert atmosphere of nitrogen, was placed 2-chloro-5-iodopyridin-4-amine (490.0 g, 1.90 mol, 1.00 equiv), TEA (974 g, 9.60 mol, 5.00 equiv), and DCM (12.30 L). This was followed by the addition of a solution of MsCl (882 g, 7.70 mol, 4.00 equiv) in DCM (7.4 L) dropwise with stirring at 0-5° C. The resulting solution was stirred for 6 hr at 0-10° C. The pH value of the solution was adjusted to 7-8 with NaHCO₃ (1 mol/L). The resulting solution was extracted with 3×5 L of dichloromethane and the organic layers combined and dried over anhydrous sodium sulfate and concentrated. This resulted in 935 g (94.6%) of N-(2-chloro-5-iodopyridin-4-yl)-N-methanesulfonylmethanesulfonamide as yellow oil.

LC-MS: (ES, m/z): [M+1]⁺=411.

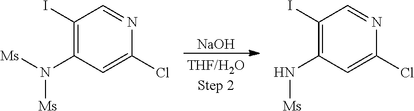

Into a 10-L 4-necked round-bottom flask purged and maintained with an inert atmosphere of nitrogen, was placed N-(2-chloro-5-iodopyridin-4-yl)-N-methanesulfonylmethanesulfonamide (935.0 g, 2.28 mol, 1.00 equiv), THE (4.70 L), H₂O (4.70 L), and NaOH (455 g, 11.4 mol, 5.00 equiv). The resulting solution was stirred for 16 hr at room temperature. The resulting mixture was concentrated. The pH of the solution was adjusted to 3-4 with citric acid (1 mol/L). The solids were collected by filtration. This resulted in 438 g (57.9%) of N-(2-chloro-5-iodopyridin-4-yl)methanesulfonamide as a white solid.

LC-MS (ES, m/z): [M+1]⁺=333.

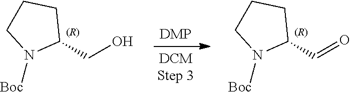

Into a 10-L 4-necked round-bottom flask purged and maintained with an inert atmosphere of nitrogen, was placed tert-butyl (2R)-2-(hydroxymethyl)pyrrolidine-1-carboxylate (530.00 g, 2.6 mol, 1.00 equiv), DCM (5.30 L), and DMP (1340 g, 3.16 mol, 1.20 equiv). The resulting solution was stirred for 6 hr at room temperature. The resulting solution was diluted with 5.3 L of H₂O. The resulting solution was extracted with 3×10 L of ethyl acetate and the organic layers combined. The resulting mixture was washed with 3×5 L of NaS₂O₃ (aq.) and 3×5 L of NaHCO₃ (aq.). The resulting mixture was washed with 3×10 L of brine. The mixture was dried over anhydrous sodium sulfate and concentrated. This resulted in 415 g (79.09%) of tert-butyl (2R)-2-formylpyrrolidine-1-carboxylate as yellow oil.

LC-MS: (ES, m/z): [M+1]⁺=200.

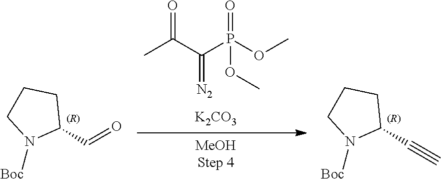

Into a 10-L 4-necked round-bottom flask purged and maintained with an inert atmosphere of nitrogen, was placed K₂CO₃ (348 g, 2.5 mol, 1.20 equiv), methanol (4.15 L), tert-butyl (2R)-2-formylpyrrolidine-1-carboxylate (415.00 g, 2.09 mol, 1.00 equiv), and dimethyl (1-diazo-2-oxopropyl)phosphonate (600 g, 3.1 mol, 1.50 equiv). The reaction mixture was stirred for 16 hr at room temperature and diluted with 4 L of H₂O. The resulting solution was extracted with 3×4 L of petroleum ether and the organic layers combined and dried over anhydrous sodium sulfate and concentrated. This resulted in 297 g (73.03%) of tert-butyl (2R)-2-ethynylpyrrolidine-1-carboxylate as yellow oil.

LC-MS: (ES, m/z): [M+1]⁺=196.

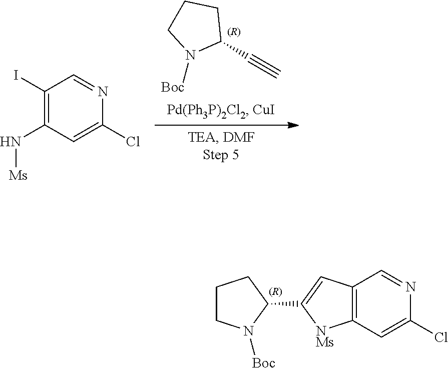

Into a 10-L 4-necked round-bottom flask purged and maintained with an inert atmosphere of nitrogen, was placed N-(2-chloro-5-iodopyridin-4-yl)methanesulfonamide (438.00 g, 1.32 mol, 1.00 equiv), TEA (533 g, 5.27 mol, 4.00 equiv), dimethylformamide (4.40 L), tert-butyl (2R)-2-ethynylpyrrolidine-1-carboxylate (283 g, 1.45 mol, 1.10 equiv), Pd(PPh₃)₂Cl₂ (46 g, 0.066 mol, 0.05 equiv), and CuI (25 g, 0.13 mol, 0.10 equiv). The reaction mixture was stirred for 6 hr at 55° C. and diluted with 4.4 L of H₂O. The resulting solution was extracted with 3×4.4 L of ethyl acetate and the organic layers combined and washed with 3×4.4 L of brine, dried over anhydrous sodium sulfate and concentrated. The residue was applied onto a silica gel column eluting with ethyl acetate/petroleum ether (1:5). This resulted in 363 g (68.9%) of tert-butyl (2R)-2-[6-chloro-1-methanesulfonylpyrrolo[3,2-c]pyridin-2-yl]pyrrolidine-1-carboxylate as a white solid.

LC-MS: (ES, m/z): [M+1]⁺=400.

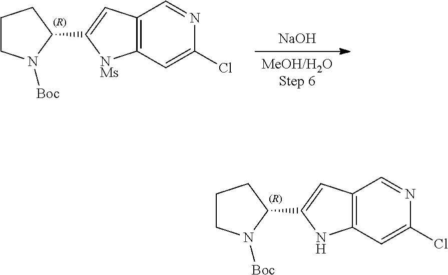

Into a 10-L 4-necked round-bottom flask purged and maintained with an inert atmosphere of nitrogen, was placed tert-butyl (2R)-2-[6-chloro-1-methanesulfonylpyrrolo[3,2-c]pyridin-2-yl]pyrrolidine-1-carboxylate (363.00 g, 0.91 mol, 1.00 equiv), MeOH (2.50 L), H₂O (1.10 L), and NaOH (109 g, 2.72 mol, 3.00 equiv). The reaction mixture was stirred for 16 hr at room temperature and concentrated. The solids were collected by filtration. This resulted in 259 g (88.67%) of tert-butyl (2R)-2-[6-chloro-1H-pyrrolo[3,2-c]pyridin-2-yl]pyrrolidine-1-carboxylate as a white solid.

LC-MS: (ES, m/z): [M+1]⁺=322.

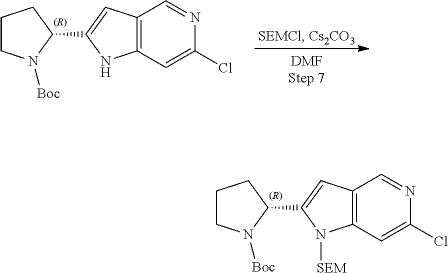

Into a 5-L 4-necked round-bottom flask purged and maintained with an inert atmosphere of nitrogen, was placed tert-butyl (2R)-2-[6-chloro-1H-pyrrolo[3,2-c]pyridin-2-yl]pyrrolidine-1-carboxylate (259.0 g, 0.80 mol, 1.00 equiv), Cs₂CO₃ (787 g, 2.4 mol, 3.00 equiv), and DMF (2.60 L). This was followed by the addition of SEMCl (161 g, 0.97 mol, 1.20 equiv) dropwise with stirring at 0-5° C. The reaction mixture was stirred for 6 hr at room temperature. The resulting solution was diluted with 2.6 L of H₂O and extracted with 3×2.6 L of ethyl acetate and the organic layers combined and washed with 3×2 L of brine. The organic layer was dried over anhydrous sodium sulfate and concentrated. The solids were collected by filtration. This resulted in 248 g (68.2%) of tert-butyl (2R)-2-(6-chloro-1-[[2-(trimethylsilyl)ethoxy]methyl]pyrrolo[3,2-c]pyridin-2-yl)pyrrolidine-1-carboxylate as a white solid.

LC-MS: (ES, m/z): [M+1]⁺=452.

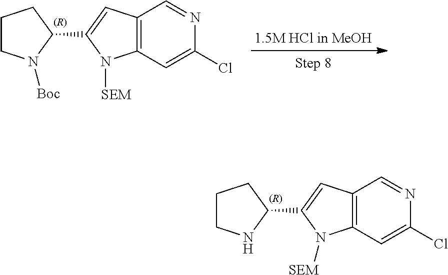

Into a 10-L 4-necked round-bottom flask purged and maintained with an inert atmosphere of nitrogen, was placed tert-butyl (2R)-2-(6-chloro-1-[[2-(trimethylsilyl)ethoxy]methyl]pyrrolo[3,2-c]pyridin-2-yl)pyrrolidine-1-carboxylate (248.00 g, 0.55 mol, 1.00 equiv), MeOH (2.40 L), and HCl (1.5 M) in MeOH (1.20 L). The reaction mixture was stirred for 12 hr at room temperature and concentrated. The resulting solution was diluted with 2.5 L of H₂O. The pH value of the solution was adjusted to 7-8 with NaHCO₃ (1 mol/L) and extracted with 3×2.5 L of dichloromethane, then the organic layers combined and concentrated. The residue was applied onto a silica gel column eluting with ethyl acetate/petroleum ether (1:2). This resulted in 177 g (91.7%) of (2R)-2-(6-chloro-1-[[2-(trimethylsilyl)ethoxy]methyl]pyrrolo[3,2-c]pyridin-2-yl)pyrrolidine as a white solid.

LC-MS (ES, m/z): [M+1]⁺=352.

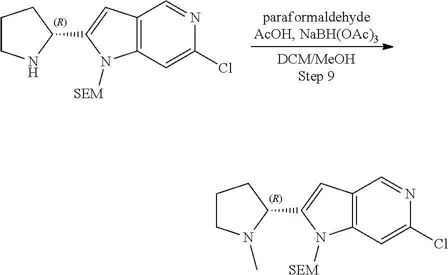

Into a 10-L 4-necked round-bottom flask purged and maintained with an inert atmosphere of nitrogen, was placed (2R)-2-(6-chloro-1-[[2-(trimethylsilyl)ethoxy]methyl]pyrrolo[3,2-c]pyridin-2-yl)pyrrolidine (177.00 g, 0.5 mol, 1.00 equiv), DCM (3.50 L), MeOH (1.77 L), paraformaldehyde (453 g, 5 mol, 10.00 equiv), and NaBH(OAc)₃ (640 g, 3 mol, 6.00 equiv). The reaction mixture was stirred for 12 hr at room temperature. The pH value of the solution was adjusted to 8-9 with NaHCO₃ (1 mol/L) and the solids were filtered out. The filtrate was extracted with 3×1.7 L of dichloromethane and the organic layers combined and concentrated. The residue was applied onto a silica gel column eluting with ethyl acetate/petroleum ether (1:3). This resulted in 129 g (70.1%) of (2R)-2-(6-chloro-1-[[2-(trimethylsilyl)ethoxy]methyl]pyrrolo[3,2-c]pyridin-2-yl)-1-methylpyrrolidine as yellow oil.

LC-MS: (ES, m/z): [M+1]⁺=366.

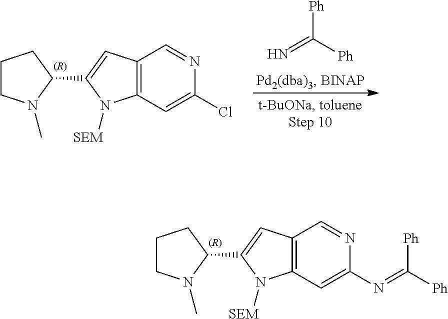

Into a 5-L 4-necked round-bottom flask purged and maintained with an inert atmosphere of nitrogen, was placed (2R)-2-(6-chloro-1-[[2-(trimethylsilyl)ethoxy]methyl]pyrrolo[3,2-c]pyridin-2-yl)-1-methylpyrrolidine (129.00 g, 0.35 mol, 1.00 equiv), toluene (2.60 L), BINAP (22 g, 0.035 mol, 0.10 equiv), t-BuONa (101 g, 1.06 mol, 3.00 equiv), Pd₂(dba)₃ (16 g, 0.017 mol, 0.05 equiv), and diphenylmethanimine (192 g, 1.06 mol, 3.00 equiv). The reaction mixture was stirred for 16 hr at 110° C. and diluted with 2.6 L of EA. The organic layer was collected and washed with 3×1 L of brine, dried over anhydrous sodium sulfate and concentrated. The residue was applied onto a silica gel column eluting with THF/PE (1:3). This resulted in 131 g (72.8%) of N-[2-[(2R)-1-methylpyrrolidin-2-yl]-1-[[2-(trimethylsilyl)ethoxy]methyl]pyrrolo[3,2-c]pyridin-6-yl]-1,1-diphenylmethanimine as yellow oil.

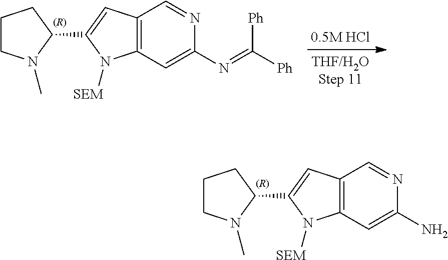

Into a 10-L 4-necked round-bottom flask purged and maintained with an inert atmosphere of nitrogen, was placed N-[2-[(2R)-1-methylpyrrolidin-2-yl]-1-[[2-(trimethylsilyl)ethoxy]methyl]pyrrolo[3,2-c]pyridin-6-yl]-1,1-diphenylmethanimine (131.00 g, 0.26 mol, 1.00 equiv), THF (6.50 L), H₂O (1.10 L), and HCl (0.5 M) (88 g, 1.28 mol, 5.00 equiv). The resulting solution was stirred for 12 hr at room temperature. The resulting solution was diluted with 2.6 L of H₂O. The resulting solution was extracted with 3×1 L of dichloromethane and the aqueous layers combined. The pH value of the solution was adjusted to 8-9 with NaHCO₃ (1 mol/L). The resulting solution was extracted with 3×2 L of dichloromethane and the organic layers combined and dried over anhydrous sodium sulfate and concentrated. The residue was applied onto a silica gel column with THF/PE (1:1). This resulted in 53.1 g (59.7%) of 2-[(2R)-1-methylpyrrolidin-2-yl]-1-[[2-(trimethylsilyl)ethoxy]methyl]pyrrolo[3,2-c]pyridin-6-amine as brown oil.

LC-MS: (ES, m/z): [M+1]⁺=347.

¹H-NMR: (300 MHz, CD₃OD, ppm): δ 8.18 (d, J=1.0 Hz, 1H), 6.68 (d, J=1.0 Hz, 1H), 6.46 (s, 1H), 5.61-5.48 (m, 2H), 3.61-3.49 (m, 3H), 3.23 (t, J=7.9 Hz, 1H), 2.46-2.35 (m, 2H), 2.33 (s, 3H), 2.02-1.84 (m, 3H), 0.90 (dd, J=8.8, 7.4 Hz, 2H), 0.0 (s, 9H)

SFC: Chiralpack IC-3 50×3.0 mm, 3 um; IPA-Hex=1:1 (20 mM NH₃); RT1.27 (>98% ee)

### Intermediate 2: (R)-2-(1-methylpiperidin-2-yl)-1-((2-(trimethylsilyl)ethoxy)methyl)-1H-pyrrolo[3,2-c]pyridin-6-amine

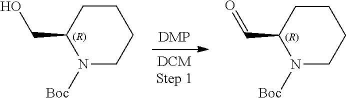

Into a 50-mL 3-necked round-bottom flask, was placed tert-butyl (2R)-2-(hydroxymethyl)piperidine-1-carboxylate (5.00 g, 23.22 mmol, 1.00 equiv), DMP (19.70 g, 46.45 mmol, 2.00 equiv), and DCM (20.00 mL). The resulting solution was stirred for 3 hr at room temperature. The reaction was then quenched by the addition of Na₂S₂O₃ (aq). The resulting solution was extracted with 2×50 mL of dichloromethane and the organic layers combined and concentrated. This resulted in 4.0 g (80.8%) of tert-butyl (2R)-2-formylpiperidine-1-carboxylate as brown oil.

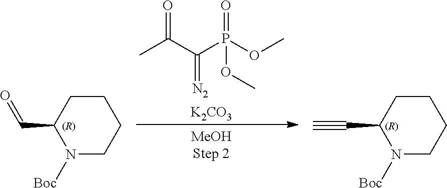

Into a 50-mL 3-necked round-bottom flask, was placed tert-butyl (2R)-2-formylpiperidine-1-carboxylate (4.00 g, 18.755 mmol, 1.00 equiv), K₂CO₃ (3.11 g, 22.506 mmol, 1.20 equiv), and MeOH (12.00 mL). This was followed by the addition of a solution of dimethyl (1-diazo-2-oxopropyl)phosphonate (5.40 g, 0.028 mmol, 1.50 equiv) in MeOH (6 mL) dropwise with stirring at 0° C. The resulting solution was stirred for 6 hr at room temperature. The resulting solution was extracted with 2×50 mL of petroleum ether and the organic layers combined and concentrated. This resulted in 2 g (51%) of tert-butyl (2R)-2-ethynylpiperidine-1-carboxylate as yellow oil.

GC-MS: (ES, m/z): [M-81]=128.

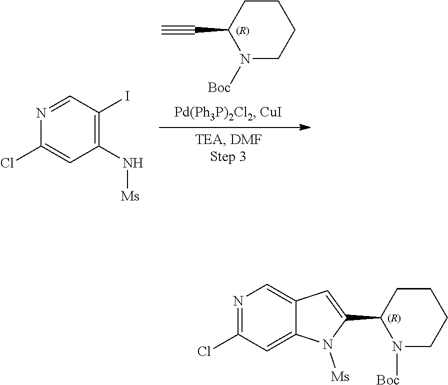

Into a 50-mL 3-necked round-bottom flask purged and maintained with an inert atmosphere of nitrogen, was placed N-(2-chloro-5-iodopyridin-4-yl)methanesulfonamide (700.0 mg, 2.1 mmol, 1.00 equiv), tert-butyl (2R)-2-ethynylpiperidine-1-carboxylate (881.11 mg, 4.210 mmol, 2.00 equiv), CuI (40.09 mg, 0.211 mmol, 0.10 equiv), TEA (852.02 mg, 8.420 mmol, 4.00 equiv), DMF (10.00 mL), and Pd(PPh₃)₂Cl₂ (295.5 mg, 0.421 mmol, 0.20 equiv). The resulting solution was stirred for 2 hr at 55° C. The solids were filtered out and the resulting mixture was concentrated. The residue was applied onto a silica gel column with ethyl acetate/petroleum ether (1:10). This resulted in 540 mg (62%) of tert-butyl (2R)-2-[6-chloro-1-methanesulfonylpyrrolo[3,2-c]pyridin-2-yl]piperidine-1-carboxylate as a brown solid.

LC-MS: (ES, m/z): [M+H]=414

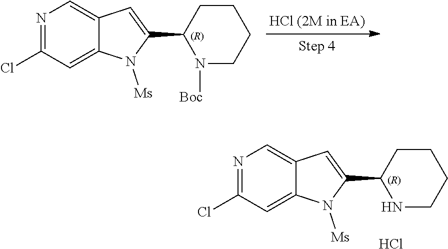

Into a 50-mL round-bottom flask, was placed tert-butyl (2R)-2-[6-chloro-1-methanesulfonylpyrrolo[3,2-c]pyridin-2-yl]piperidine-1-carboxylate (430.00 mg) and HCl (gas) in ethyl acetate (10.00 mL). The resulting solution was stirred for 6 hr at room temperature. The resulting mixture was concentrated. This resulted in 380 mg of (2R)-2-[6-chloro-1-methanesulfonylpyrrolo[3,2-c]pyridin-2-yl]piperidine hydrochloride as a brown solid.

LC-MS: (ES, m/z): [M+H—HCl]=314.

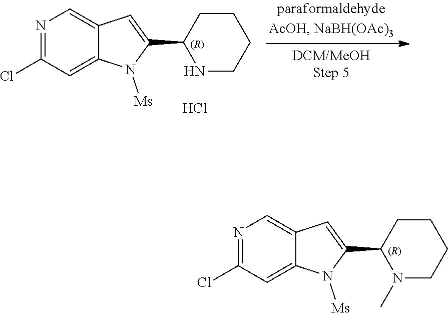

Into a 100-mL 3-necked round-bottom flask, was placed (2R)-2-[6-chloro-1-methanesulfonylpyrrolo[3,2-c]pyridin-2-yl]piperidine hydrochloride (380.0 mg, 1.089 mmol, 1.00 equiv), DCM (20.00 mL), MeOH (10.00 mL), paraformaldehyde (488.63 mg, 5.43 mmol, 5.00 equiv), and NaBH(OAc)₃ (2299.37 mg, 10.85 mmol, 10.00 equiv). The resulting solution was stirred for 12 hr at room temperature. The reaction was then quenched by the addition of 20 mL of water. The resulting solution was extracted with 2×30 mL of dichloromethane and the organic layers combined and concentrated. The residue was applied onto a silica gel column with ethyl acetate/petroleum ether (1:1). This resulted in 201 mg (56.5%) of (2R)-2-[6-chloro-1-methanesulfonylpyrrolo[3,2-c]pyridin-2-yl]-1-methylpiperidine as a white solid.

LC-MS: (ES, m/z): [M+H]=328.

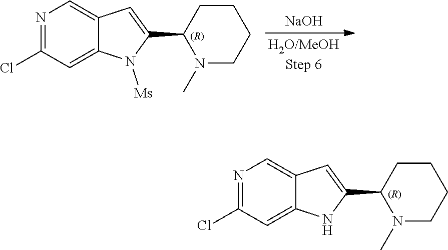

Into a 50-mL round-bottom flask, was placed (2R)-2-[6-chloro-1-methanesulfonylpyrrolo[3,2-c]pyridin-2-yl]-1-methylpiperidine (185.00 mg, 0.564 mmol, 1.00 equiv), NaOH (67.71 mg, 0.000 mmol, 3.00 equiv), and H₂O (1.00 mL), MeOH (5.00 mL). The resulting solution was stirred for 2 hr at room temperature. The resulting solution was extracted with 2×20 mL of ethyl acetate and the organic layers combined and concentrated. This resulted in 120 mg (85.2%) of (2R)-2-[6-chloro-1H-pyrrolo[3,2-c]pyridin-2-yl]-1-methylpiperidine as a brown solid.

LC-MS: (ES, m/z): [M+H]=250.

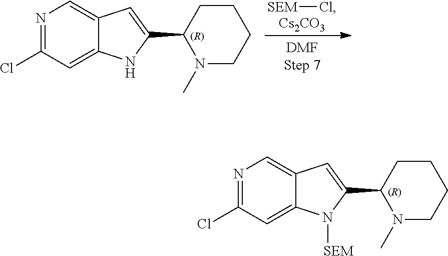

Into a 50-mL 3-necked round-bottom flask, was placed (2R)-2-[6-chloro-1H-pyrrolo[3,2-c]pyridin-2-yl]-1-methylpiperidine (120.0 mg, 0.480 mmol, 1.00 equiv), Cs₂CO₃ (469.7 mg, 1.44 mmol, 3.00 equiv), DMF (5.00 mL), SEM-Cl (120.16 mg, 0.720 mmol, 1.50 equiv). The resulting solution was stirred for 2 hr at room temperature. The solids were filtered, and the resulting solution extracted with 2×20 mL of ethyl acetate. The organic layers were combined and concentrated. The residue was applied onto a silica gel column eluting with ethyl acetate/petroleum ether (1:2). This resulted in 85 mg (46.6%) of (2R)-2-(6-chloro-1-[[2-(trimethylsilyl)ethoxy]methyl]pyrrolo[3,2-c]pyridin-2-yl)-1-methylpiperidine as light brown oil.

LC-MS: (ES, m/z): [M+H]=380.

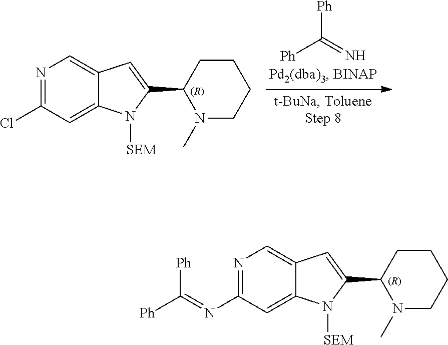

Into a 8-mL sealed tube purged and maintained with an inert atmosphere of nitrogen, was placed 2-(6-chloro-1-[[2-(trimethylsilyl)ethoxy]methyl]-octahydropyrrolo[3,2-c]pyridin-2-yl)-1-methylpiperidine (80.00 mg, 0.206 mmol, 1.00 equiv), diphenylmethanimine (112.09 mg, 0.618 mmol, 3.00 equiv), t-BuONa (59.43 mg, 0.618 mmol, 3.00 equiv), toluene (3.00 mL), Pd₂(dba)₃·CHCl₃ (23.71 mg, 0.041 mmol, 0.20 equiv), and BINAP (51.35 mg, 0.082 mmol, 0.40 equiv). The resulting solution was stirred for 5 hr at 100° C. The resulting mixture was concentrated. This resulted in 100 mg (crude) of N-[2-[(2R)-1-methylpiperidin-2-yl]-1-[[2-(trimethylsilyl)ethoxy]methyl]pyrrolo[3,2-c]pyridin-6-yl]-1,1-diphenylmethanimine as brown oil.

LC-MS: (ES, m/z): [M+H]=525.

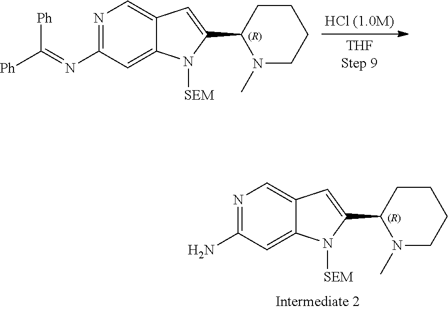

Into a 50-mL round-bottom flask, was placed N-[2-[(2R)-1-methylpiperidin-2-yl]-1-[[2-(trimethylsilyl)ethoxy]methyl]pyrrolo[3,2-c]pyridin-6-yl]-1,1-diphenylmethanimine (100.00 mg, 0.191 mmol, 1.00 equiv), THE (5.00 mL), and HCl (5.00 mL). The resulting solution was stirred for 16 hr at room temperature and extracted with 2×20 mL of ethyl acetate, then the organic layers combined and concentrated. The residue was applied onto a silica gel column eluting with ethyl acetate/petroleum ether (1:1). This resulted in 44 mg (34% for two steps) of 2-[(2R)-1-methylpiperidin-2-yl]-1-[[2-(trimethylsilyl)ethoxy]methyl]pyrrolo[3,2-c]pyridin-6-amine as a brown solid. The product was further purified by SFC with the following conditions (Column: Lux 5 m Amylose-1, 5×25 cm, 10 m; Mobile Phase A: CO₂, Mobile Phase B: IPA (0.5% 2M NH₃-MeOH); Flow rate: 160 mL/min; Gradient: isocratic 40% B; Column Temperature (° C.): 35; Back Pressure (bar): 100; Wave Length: 220 nm; RT1(min): 4.47; RT2(min): 5.89; Sample Solvent: ACN; Injection Volume: 2 mL and the major enantiomer collected to obtain material >98% ee.

LC-MS: (ES, m/z): [M+H]=361

¹H-NMR (300 MHz, Methanol-d₄, ppm): δ 8.23-8.17 (m, 1H), 6.68 (s, 1H), 6.47 (s, 1H), 3.56 (t, J=8.2 Hz, 2H), 3.08 (d, J=11.9 Hz, 1H), 2.24 (d, J=14.1 Hz, 1H), 2.15 (s, 3H), 1.97-1.68 (m, 5H), 1.48 (d, J=10.5 Hz, 1H), 1.17 (d, J=6.2 Hz, 3H), 0.91 (t, J=8.1 Hz, 2H), −0.22 (s, 9H).

### Acid 1: 5-(1-(tetrahydro-2H-pyran-2-yl)-1H-pyrazol-4-yl)picolinic acid

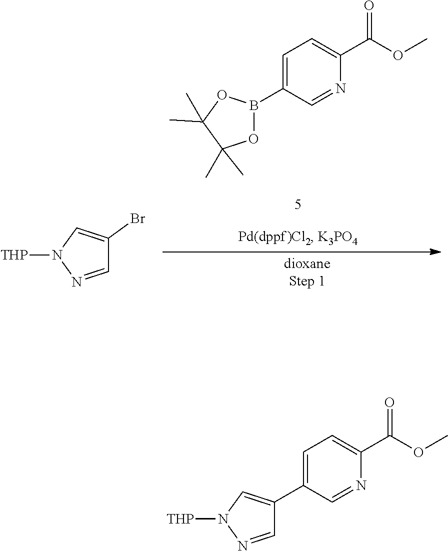

Into a 40-mL vial purged and maintained with an inert atmosphere of nitrogen, was placed methyl 5-(4,4,5,5-tetramethyl-1,3,2-dioxaborolan-2-yl)pyridine-2-carboxylate (1.00 g, 3.801 mmol, 1.00 equiv), dioxane (20.00 mL), 4-bromo-1-(oxan-2-yl)pyrazole (0.97 g, 4.181 mmol, 1.1 equiv), Pd(dppf)Cl₂ (0.28 g, 0.380 mmol, 0.1 equiv), and K₃PO₄ (2.42 g, 11.403 mmol, 3 equiv). The reaction mixture was stirred for 5 h at 100° C. in an oil bath under nitrogen atmosphere and concentrated under vacuum. The residue was diluted with 30 mL of H₂O and extracted with 3×20 mL of ethyl acetate. The organic layers combined and dried over anhydrous sodium sulfate and concentrated under vacuum. The residue was applied onto a silica gel column eluting with ethyl acetate/petroleum ether (1:1). This resulted in 800 mg (73.3%) of methyl 5-[1-(oxan-2-yl)pyrazol-4-yl]pyridine-2-carboxylate as a yellow solid.

LC-MS: (ES, m/z): [M+H]⁺=288

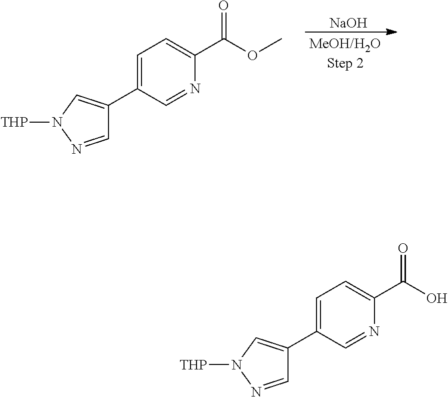

Into a 50-mL round-bottom flask, was placed methyl 5-[1-(oxan-2-yl)pyrazol-4-yl]pyridine-2-carboxylate (800 mg, 2.784 mmol, 1.00 equiv), CH₃OH (16 mL), H₂O (5 mL), and sodium hydroxide (330 mg, 8.251 mmol, 2.96 equiv). The resulting solution was stirred for 16 h at room temperature and concentrated under vacuum. The residue was diluted with 30 mL of H₂O and extracted with 2×20 mL of ethyl acetate, then the aqueous layers combined. The pH value of the solution was adjusted to 3 with HCl (3 mol/L). The resulting solids were collected by filtration to give 600 mg (78.85%) of 5-(1-(tetrahydro-2H-pyran-2-yl)-1H-pyrazol-4-yl)picolinic acid as a light yellow solid.

LC-MS: (ES, m/z): [M+H]⁺=274

### Acid 2: 4-[5-(methoxymethyl)-1-{[2-(trimethylsilyl)ethoxy]methyl}pyrazol-4-yl]benzoic acid

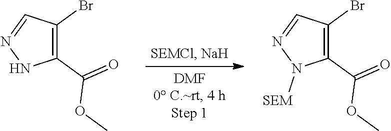

A solution of methyl 4-bromo-2H-pyrazole-3-carboxylate (3.0 g, 14.633 mmol, 1 equiv) in DMF (30 mL) was treated with NaH (900 mg, 37.503 mmol, 2.56 equiv) for 10 min at 0° C. under nitrogen atmosphere followed by the addition of [2-(chloromethoxy)ethyl]trimethylsilane (3.7 g, 22.193 mmol, 1.52 equiv) dropwise at 0° C. The mixture was stirred 4 h at RT. The reaction was quenched by the addition of water (20 mL) at 0° C. The resulting mixture was extracted with EA (50 mL×3). The combined organic layers were washed with NaCl(aq) (50 mL×3), dried over anhydrous Na₂SO₄ and concentrated under reduced pressure. The residue was purified by silica gel column chromatography, eluted with EA/PE=3/1 to afford methyl 4-bromo-2-{[2-(trimethylsilyl)ethoxy]methyl}pyrazole-3-carboxylate (3.5 g, 64.20%) as a yellow solid.

LC-MS (ES, m/z): [M+1]⁺=335.1, 337.1

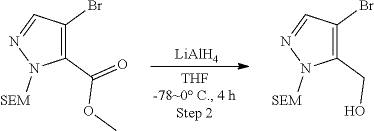 [SMILES: COC(=O)c1c(Br)cnn1COCC[Si](C)(C)C ; "LiAlH4" ; C[Si](C)(C)CCOCn1ncc(Br)c1CO]

To a solution of LiAlH₄ (400 mg, 10.540 mmol, 2.94 equiv) in THF (10 mL, 123.428 mmol, 34.48 equiv) under nitrogen atmosphere, methyl 4-bromo-2-{[2-(trimethylsilyl)ethoxy]methyl}pyrazole-3-carboxylate (1.2 g, 3.579 mmol, 1 equiv) in THE was added dropwise at −78° C. The mixture was stirred 4 h at −78° C.-0° C. The reaction was quenched by the addition of water/NaOH at −30° C. and filtered, then the filter cake was washed with EA. The filtrate was extracted with EA (20 mL×3). The combined organic layers were washed with NaCl(aq), dried over anhydrous Na₂SO₄ and concentrated under reduced pressure. The residue was purified by silica gel column chromatography, eluting with EA/PE=1/2 to afford (4-bromo-2-{[2-(trimethylsilyl)ethoxy]methyl}pyrazol-3-yl)methanol (270 mg, 22%) as a yellow oil.

LC-MS (ES, m/z): [M+1]⁺=306.9, 308.9

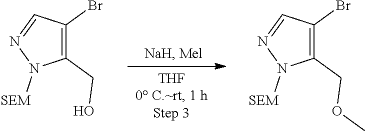 [SMILES: C[Si](C)(C)CCOCn1ncc(Br)c1CO ; COCc1c(Br)cnn1COCC[Si](C)(C)C]

To a solution of (4-bromo-2-{[2-(trimethylsilyl)ethoxy]methyl}pyrazol-3-yl)methanol (250 mg, 0.814 mmol, 1 equiv) in THE was added sodium hydride (60% in oil, NaH (25 mg, 1.042 mmol, 1.28 equiv) mg) at 0° C. The mixture was stirred for 15 min at 0° C. CH₃I (200 mg, 1.409 mmol, 1.73 equiv) was added and the mixture warmed to RT and stirred for 1 h. The reaction mixture was quenched by water and extracted with DCM (3×25 mL). The residue was purified by silica gel column chromatography, eluted with EA/PE=1/4 to afford 4-bromo-5-(methoxymethyl)-1-{[2-(trimethylsilyl)ethoxy]methyl}pyrazole (200 mg, 68.86%) as a yellow oil.

LC-MS (ES, m/z): [M+1]⁺=321.2, 323.2

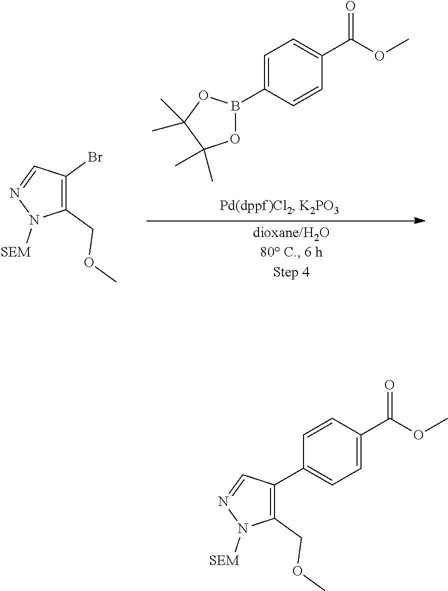

To a solution of 4-bromo-5-(methoxymethyl)-1-{[2-(trimethylsilyl)ethoxy]methyl}pyrazole (200 mg, 0.622 mmol, 1 equiv) and methyl 4-(4,4,5,5-tetramethyl-1,3,2-dioxaborolan-2-yl)benzoate (250 mg, 0.954 mmol, 1.53 equiv) in dioxane (8 mL, 94.432 mmol, 151.70 equiv), and water (0.8 mL, 44.407 mmol, 71.34 equiv) were added K₂CO₃ (250 mg, 1.809 mmol, 2.91 equiv) and Pd(dppf)Cl₂ (20 mg, 0.027 mmol, 0.04 equiv). After stirring for 6 at 80° C. under a nitrogen atmosphere, the resulting mixture was concentrated under reduced pressure. The residue was purified by Prep-TLC/silica gel column chromatography, eluting with EA/PE=1/3 to afford methyl 4-[5-(methoxymethyl)-1-{[2-(trimethylsilyl)ethoxy]methyl}pyrazol-4-yl]benzoate (150 mg, 57.6%) as a yellow solid.

LC-MS (ES, m/z): [M+1]⁺=377.2

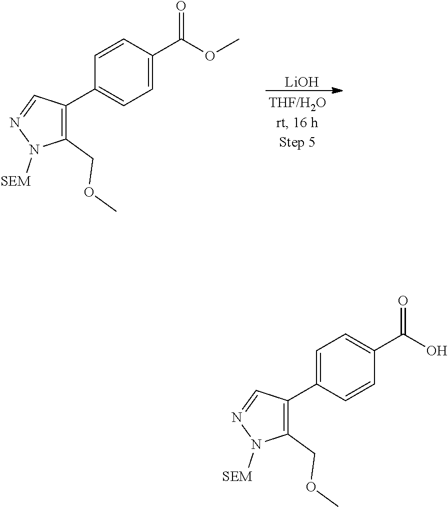 [SMILES: COCc1c(-c2ccc(C(=O)OC)cc2)cnn1COCC[Si](C)(C)C ; "LiOH" ; COCc1c(-c2ccc(C(=O)O)cc2)cnn1COCC[Si](C)(C)C]

To a solution of methyl 4-[5-(methoxymethyl)-1-{[2-(trimethylsilyl)ethoxy]methyl}pyrazol-4-yl]benzoate (150 mg, 0.398 mmol, 1 equiv) in THF (8 mL, 98.742 mmol, 247.86 equiv) and water (1 mL, 55.509 mmol, 139.34 equiv), LiOH (100 mg, 4.175 mmol, 10.48 equiv) was added. The mixture was stirred 16 hours at RT and acidified to pH 5 with oxalic acid. The resulting mixture was extracted with EA (20 mL×3). The combined organic layers were washed with NaCl(aq), dried over anhydrous Na₂SO₄ and the filtrate was concentrated under reduced pressure to afford 4-[5-(methoxymethyl)-1-{[2-(trimethylsilyl)ethoxy]methyl}pyrazol-4-yl]benzoic acid (100 mg, 58.86%) as a yellow solid.

LC-MS (ES, m/z): [M+1]⁺=363.2

### Example 1: N-(2-methyl-1H-pyrrolo[3,2-c]pyridin-6-yl)-5-(1H-pyrazol-4-yl)picolinamide (Sample 1)

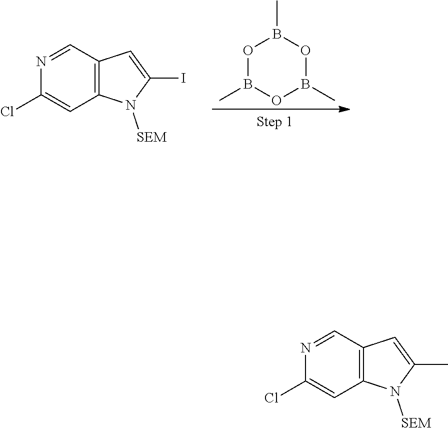

To a solution of 6-chloro-2-iodo-1-{[2-(trimethylsilyl)ethoxy]methyl}pyrrolo[3,2-c]pyridine (550 mg, 1.346 mmol, 1.00 equiv) and trimethyl-1,3,5,2,4,6-trioxatriborinane (1.69 g, 13.460 mmol, 10 equiv) in DMF (2 mL) was added K₂CO₃ (557.91 mg, 4.038 mmol, 3 equiv) and Pd(dppf)Cl₂·CH₂Cl₂ (109.62 mg, 0.135 mmol, 0.1 equiv). After stirring for 60 h at 80° C. under a nitrogen atmosphere, the resulting mixture was concentrated under reduced pressure. The residue was purified by silica gel column chromatography, eluted with PE/EA (5:1) to afford 6-chloro-2-methyl-1-{[2-(trimethylsilyl)ethoxy]methyl}pyrrolo[3,2-c]pyridine (296 mg, 74.10%) as a yellow solid.

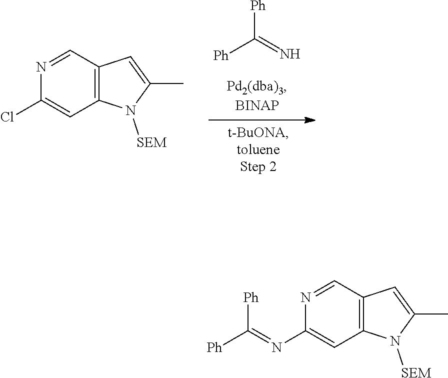 [SMILES: Cc1cc2cnc(Cl)cc2n1COCC[Si](C)(C)C ; N=C(c1ccccc1)c1ccccc1 ; Cc1cc2cnc(N=C(c3ccccc3)c3ccccc3)cc2n1COCC[Si](C)(C)C]

To a solution of 6-chloro-2-methyl-1-{[2-(trimethylsilyl)ethoxy]methyl}pyrrolo[3,2-c]pyridine (290 mg, 0.977 mmol, 1.00 equiv) and diphenylmethanimine (531.13 mg, 2.931 mmol, 3 equiv) in toluene (2 mL) and BINAP (121.65 mg, 0.195 mmol, 0.2 equiv) were added t-BuONa (281.64 mg, 2.931 mmol, 3 equiv) and Pd₂(dba)₃CHCl₃ (101.11 mg, 0.098 mmol, 0.10 equiv). After stirring for 16 h at 100° C. under a nitrogen atmosphere, the resulting mixture was cooled and concentrated under reduced pressure. The reaction was diluted with water at room temperature. The resulting mixture was extracted with CH₂Cl₂ (2×50 mL). The combined organic layers were dried over anhydrous Na₂SO₄. After filtration, the filtrate was concentrated under reduced pressure. This resulted in N-(2-methyl-1-{[2-(trimethylsilyl)ethoxy]methyl}pyrrolo[3,2-c]pyridin-6-yl)-1,1-diphenylmethanimine (300 mg, 41.72%) as a brown oil. The crude product/resulting mixture was used in the next step directly without further purification.

LC-MS: (ES, m/z): [M+H]=442

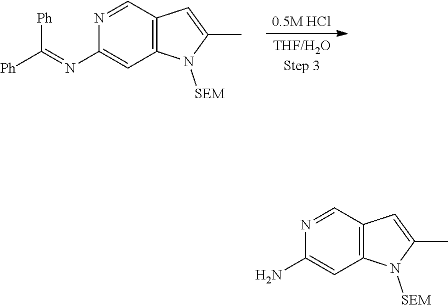 [SMILES: Cc1cc2cnc(N=C(c3ccccc3)c3ccccc3)cc2n1COCC[Si](C)(C)C ; Cc1cc2cnc(N)cc2n1COCC[Si](C)(C)C]

To a stirred solution of N-(2-methyl-1-{[2-(trimethylsilyl)ethoxy]methyl}pyrrolo[3,2-c]pyridin-6-yl)-1,1-diphenylmethanimine (300 mg, 0.408 mmol, 1.00 equiv, 60%) in THE (5 mL) was added HCl 1.0M (2.45 mL, 2.448 mmol, 6 equiv) dropwise at room temperature. The resulting mixture was stirred for 16 h at room temperature and extracted with THE (2×30 mL). The aqueous layer was basified to pH 7 with saturated NaHCO₃ (aq.) and extracted with CH₂Cl₂ (2×30 mL). The combined organic layers were washed with brine (2×20 mL) and dried over anhydrous Na₂SO₄. The filtrate was concentrated under reduced pressure. This resulted in 2-methyl-1-{[2-(trimethylsilyl)ethoxy]methyl}pyrrolo[3,2-c]pyridin-6-amine (180 mg, crude) as a brown solid.

LC-MS: (ES, m/z): [M+H]=278

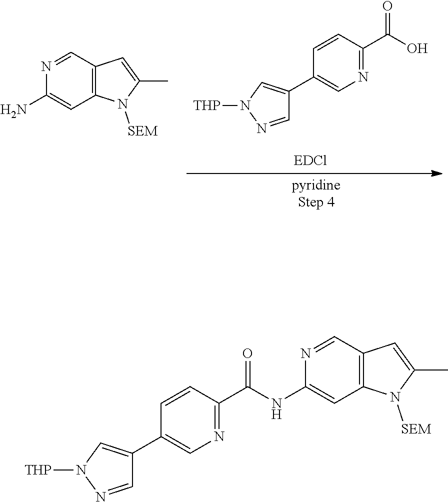

To a stirred solution of 2-methyl-1-{[2-(trimethylsilyl)ethoxy]methyl}pyrrolo[3,2-c]pyridin-6-amine (60 mg, 0.216 mmol, 1.00 equiv) and 5-(1-(tetrahydro-2H-pyran-2-yl)-1H-pyrazol-4-yl)picolinic acid (Acid 1, 59.10 mg, 0.216 mmol, 1.0 equiv) in pyridine (1 mL) was added EDCI (82.92 mg, 0.432 mmol, 2.0 equiv) at room temperature. The resulting mixture was stirred for 16 h at room temperature. The resulting mixture was diluted with water (30 mL) and extracted with EtOAc (2×50 mL). The combined organic layers were washed with brine (2×20 mL), dried over anhydrous Na₂SO₄, and concentrated under reduced pressure resulting in N-(2-methyl-1-{[2-(trimethylsilyl)ethoxy]methyl}pyrrolo[3,2-c]pyridin-6-yl)-5-[1-(oxan-2-yl)pyrazol-4-yl]pyridine-2-carboxamide (120 mg, crude) as a brown solid.

LC-MS: (ES, m/z): [M+H]=533

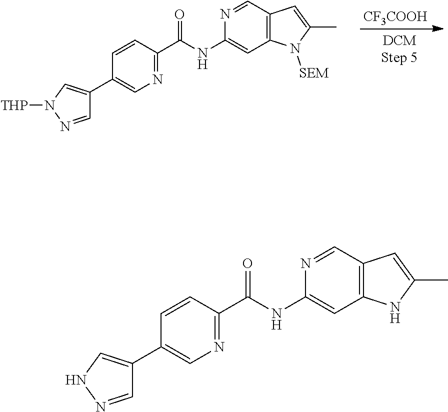

To a stirred solution of N-(2-methyl-1-{[2-(trimethylsilyl)ethoxy]methyl}pyrrolo[3,2-c]pyridin-6-yl)-5-[1-(oxan-2-yl)pyrazol-4-yl]pyridine-2-carboxamide (120 mg, crude) in DCM (1 mL) was added CF₃COOH (0.80 mL) dropwise at room temperature. The resulting mixture was stirred for 16 h at room temperature and concentrated under vacuum. The residue was basified to pH 7 with NH₄₀H (aq.). The crude product was purified by Prep-HPLC with the following conditions: Column, XBridge Prep C18 OBD Column, 5 μm, 19×150 mm; mobile phase, Water (0.05% NH₃H₂O) and ACN (41% Phase B up to 54% in 7 min); Detector, UV 254 nm) to afford N-{2-methyl-1H-pyrrolo[3,2-c]pyridin-6-yl}-5-(1H-pyrazol-4-yl)pyridine-2-carboxamide (17.8 mg) as a light yellow solid.

LC-MS: (ES, m/z): [M+H]=319

H-NMR (400 MHz, DMSO-d₆, ppm) δ 13.22 (s, 1H), 11.39 (s, 1H), 10.26 (s, 1H), 9.05 (d, J=0.8 Hz, 1H), 8.46 (s, 1H), 8.35-8.18 (m, 4H), 8.18 (d, J=8.0 Hz, 1H), 6.24 (s, 1H), 2.40 (s, 3H).

### Example 2: (R)-N-(2-(1-methylpyrrolidin-2-yl)-1H-pyrrolo[3,2-c]pyridin-6-yl)-5-(1H-pyrazol-4-yl)picolinamide (Sample 2)

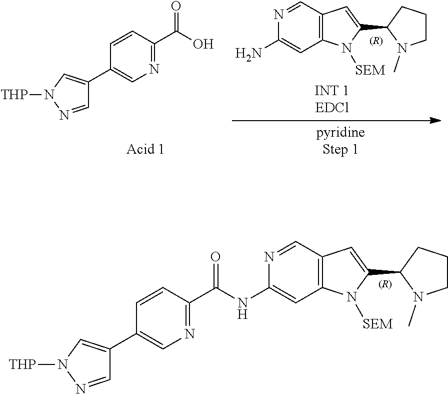

Into a 8-mL vial purged and maintained with an inert atmosphere of nitrogen, was placed 5-(1-(tetrahydro-2H-pyran-2-yl)-1H-pyrazol-4-yl)picolinic acid (Acid 1, 80 mg, 0.293 mmol, 1.00 equiv), pyridine (4 mL), EDCI (84.17 mg, 0.440 mmol, 1.5 equiv) and 2-[(2R)-1-methylpyrrolidin-2-yl]-1-{[2-(trimethylsilyl)ethoxy]methyl}pyrrolo[3,2-c]pyridin-6-amine (Intermediate 1, 101.44 mg, 0.293 mmol, 1 equiv). The resulting solution was stirred for 16 h at room temperature and concentrated under vacuum. The residue was diluted with 20 mL of H₂O and extracted with 3×10 mL of ethyl acetate, then the organic layers combined and washed with 2×10 mL of brine. The organic layer was dried over anhydrous sodium sulfate and concentrated under vacuum. This resulted in 130 mg (73.79) of N-{2-[(2R)-1-methylpyrrolidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide as brown oil.

LC-MS: (ES, m/z): [M+H]⁺=602

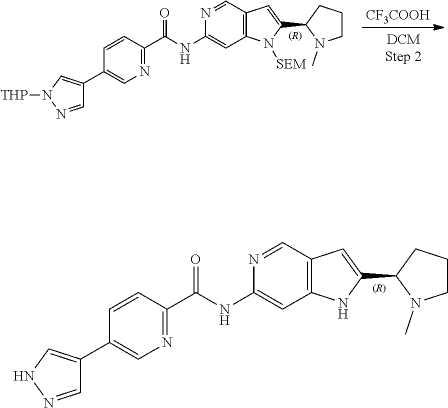

Into a 50-mL round-bottom flask, was placed N-{2-[(2R)-1-methylpyrrolidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide (130 mg, 0.216 mmol, 1.00 equiv), DCM (4.00 mL), and CF₃COOH (4.00 mL). The resulting solution was stirred for 16 h at room temperature and concentrated under vacuum. The resulting solution was diluted with 4 mL of DMF. The pH of the mixture was adjusted to 8 with NH₃/H₂O. The crude product was purified by Prep-HPLC with the following conditions Column, XBridge Shield RP18 OBD Column, 5 μm, 19×150 mm; mobile phase, Water (0.05% NH₃H₂O) and ACN (15% Phase B up to 31% in 7 min); Detector, UV 254 nm. This resulted in 22.2 mg (26.53%) of N-{2-[(2R)-1-methylpyrrolidin-2-yl]-1H-pyrrolo[3,2-c]pyridin-6-yl}-5-(1H-pyrazol-4-yl)pyridine-2-carboxamide as a yellow solid.

LC-MS: (ES, m/z): [M+H]⁺=388

¹H-NMR: (300 MHz, Methanol-d₄, ppm) δ 8.99 (s, 1H), 8.51 (d, J=0.9 Hz, 1H), 8.38 (s, 1H), 8.23 (m, 4H), 6.52 (s, 1H), 3.43 (t, J=7.8 Hz, 1H), 3.24-3.19 (m, 1H), 2.41-2.27 (m, 5H), 1.89-2.07 (m, 3H).

### Example 3: (S)-N-(2-(1-methylpyrrolidin-2-yl)-1H-pyrrolo[3,2-c]pyridin-6-yl)-5-(1H-pyrazol-4-yl)picolinamide (Sample 3)

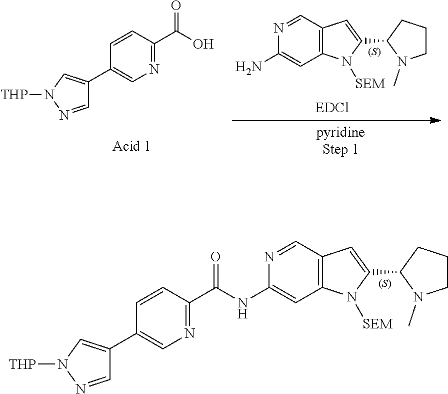

Into a 8 mL vial were added 5-(1-(tetrahydro-2H-pyran-2-yl)-1H-pyrazol-4-yl)picolinic acid (Acid 1, 60 mg, 0.220 mmol, 1.00 equiv), pyridine (4 mL), 2-[(2S)-1-methylpyrrolidin-2-yl]-1-{[2-(trimethylsilyl)ethoxy]methyl}pyrrolo[3,2-c]pyridin-6-amine (Prepared according to Intermediate 1 using tert-butyl (2S)-2-(hydroxymethyl)pyrrolidine-1-carboxylate in Step 3, 76.08 mg, 0.220 mmol, 1 equiv), and EDCI (63.13 mg, 0.330 mmol, 1.5 equiv) at room temperature. The resulting solution was stirred for 16 h at room temperature and concentrated under vacuum. The reaction mixture was diluted with 20 mL of H₂O and extracted with 3×10 mL of ethyl acetate. The organic layers combined, then washed with 2×10 mL of brine. The solution was dried over anhydrous sodium sulfate and concentrated under vacuum. This resulted in N-{2-[(2S)-1-methylpyrrolidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide (80 mg, crude) as brown oil.

LC-MS: (ES, m/z): [M+H]⁺=602

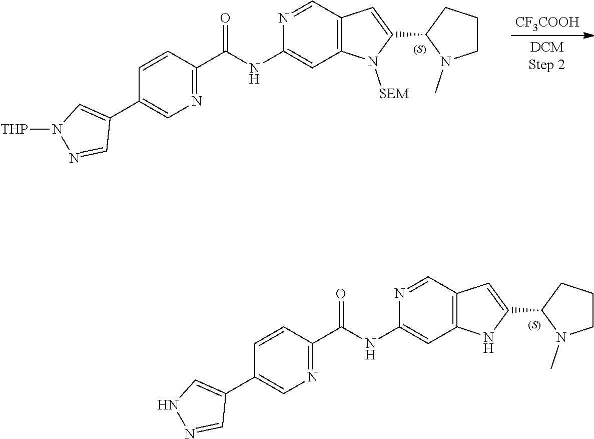

Into a 50-mL round-bottom flask, was placed N-{2-[(2S)-1-methylpyrrolidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide (80 mg), DCM (4 mL), and CF₃COOH (4 mL). The resulting solution was stirred for 16 h at room temperature and concentrated under vacuum. The residue was diluted with 4 mL of DMF. The pH value of the solution was adjusted to 8 with NH₃/H₂O. The crude product was purified by Prep-HPLC with the following conditions: Column, XBridge Shield RP18 OBD Column, 5 μm, 19×150 mm; mobile phase, Water (0.05% NH₃H₂O) and ACN (15% Phase B up to 31% in 7 min); Detector, UV 254 nm. This resulted in N-{2-[(2S)-1-methylpyrrolidin-2-yl]-1H-pyrrolo[3,2-c]pyridin-6-yl}-5-(1H-pyrazol-4-yl)pyridine-2-carboxamide (7.8 mg, 15.14%) as a light yellow solid.

LC-MS: (ES, m/z): [M+H]⁺=388

¹H-NMR: (300 MHz, Methanol-d₄, ppm) δ 8.98 (s, 1H), 8.49 (s, 1H), 8.38 (s, 1H), 8.25-8.20 (m, 4H), 6.51 (s, 1H), 3.43 (t, J=7.8 Hz, 1H), 3.28-3.16 (m, 1H), 2.43-2.34 (m, 1H), 2.29 (s, 3H), 2.30-2.21 (m, 1H), 2.09-1.94 (m, 3H).

### Example 4: (R)-2-fluoro-N-(2-(1-methylpyrrolidin-2-yl)-1H-pyrrolo[3,2-c]pyridin-6-yl)-4-(1H-pyrazol-4-yl)benzamide (Sample 4)

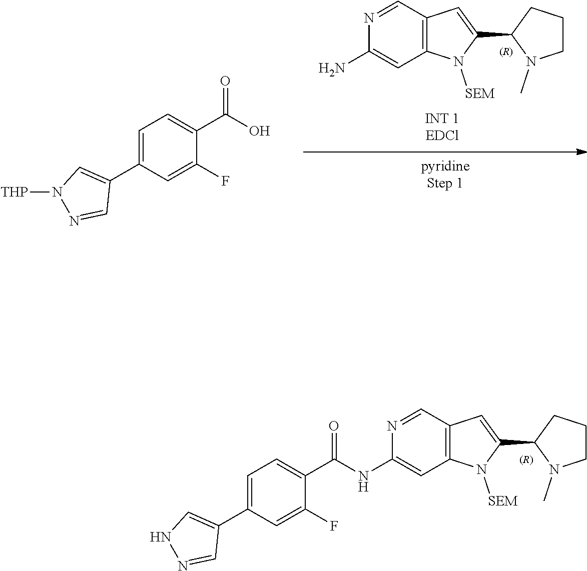

Into a 8-mL vial purged and maintained with an inert atmosphere of nitrogen, was placed 2-fluoro-4-[1-(oxan-2-yl)pyrazol-4-yl]benzoic acid (Prepared as for Acid 1, using Benzoic acid, 2-fluoro-4-(4,4,5,5-tetramethyl-1,3,2-dioxaborolan-2-yl)-, methyl ester and -bromo-1-(oxan-2-yl)pyrazole, 100 mg, 0.344 mmol, 1.00 equiv), pyridine (4 mL), and 2-[(2R)-1-methylpyrrolidin-2-yl]-1-{[2-(trimethylsilyl)ethoxy]methyl}pyrrolo[3,2-c]pyridin-6-amine (Intermediate 1, 95.50 mg, 0.275 mmol, 0.8 equiv) and EDCI (198.11 mg, 1.032 mmol, 3 equiv). The resulting solution was stirred for 16 h at room temperature and concentrated under vacuum. The resulting solution was diluted with 20 mL of H₂O and extracted with 3×10 mL of ethyl acetate, then the organic layers combined. The solution was washed with 2×10 mL of brine and dried over anhydrous sodium sulfate and concentrated under vacuum. This resulted in 2-fluoro-N-{2-[(2R)-1-methylpyrrolidin-2-yl]-1-{[2-(trimethylsilyl)ethoxy]methyl}pyrrolo[3,2-c]pyridin-6-yl}-4-(1H-pyrazol-4-yl)benzamide (100 mg, crude) as brown oil.

LC-MS: (ES, m/z): [M+H]⁺=535

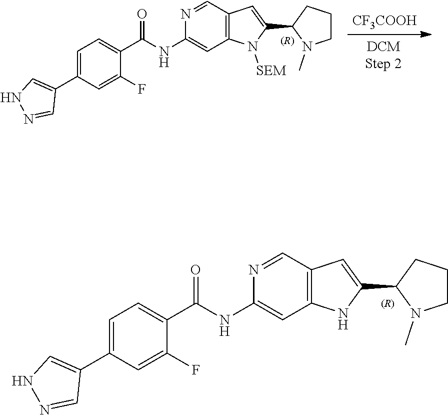

Into a 50-mL round-bottom flask, was placed 2-fluoro-N-{2-[(2R)-1-methylpyrrolidin-2-yl]-1-{[2-(trimethylsilyl)ethoxy]methyl}pyrrolo[3,2-c]pyridin-6-yl}-4-(1H-pyrazol-4-yl)benzamide (100 mg, crude), DCM (4.00 mL), and CF₃COOH (4.00 mL). The resulting solution was stirred for 20 h at room temperature and concentrated under vacuum. The residue was diluted with 4 mL of DMF and the pH value of the solution was adjusted to 8 with NH₃H₂O. The crude product (60 mg) was purified by Prep-HPLC with the following conditions: Column, XBridge Shield RP18 OBD Column, 5 μm, 19×150 mm; mobile phase, Water (0.05% NH₃H₂O) and ACN (15% Phase B up to 31% in 7 min); Detector, UV 254 nm. This resulted in 2-fluoro-N-{2-[(2R)-1-methylpyrrolidin-2-yl]-1H-pyrrolo[3,2-c]pyridin-6-yl}-4-(1H-pyrazol-4-yl)benzamide (19.4 mg) as a white solid.

LC-MS: (ES, m/z): [M+H]⁺=405

¹H-NMR: (300 MHz, Methanol-d₄, ppm) δ 8.49 (d, J=0.9 Hz, 1H), 8.29 (s, 1H), 8.21-8.05 (m, 2H), 7.97 (t, J=8.1 Hz, 1H), 7.65-7.51 (m, 2H), 6.51 (s, 1H), 3.43 (t, J=7.9 Hz, 1H), 3.25-3.20 (m, 1H), 2.43-2.24 (m, 5H), 2.07-1.94 (m, 3H).

F-NMR: (282 MHz, Methanol-d₄, ppm) 6-114.965

### Example 5: (R)-N-(2-(1-methylpyrrolidin-2-yl)-1H-pyrrolo[3,2-c]pyridin-6-yl)-4-(1H-pyrazol-4-yl)benzamide (Sample 5)

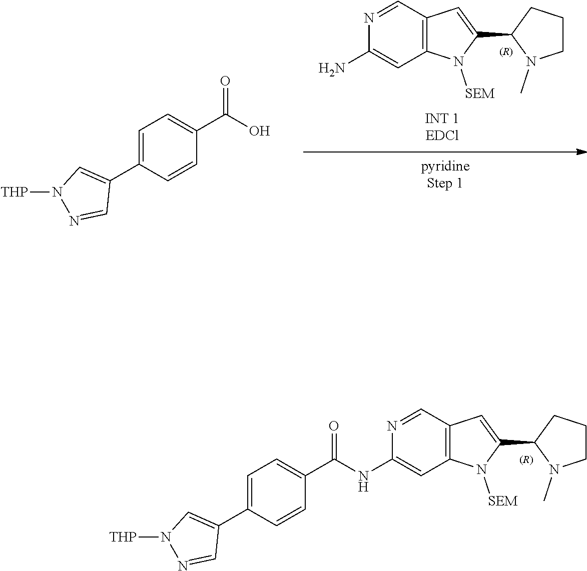

To a stirred solution of 4-[1-(oxan-2-yl)pyrazol-4-yl]benzoic acid (Prepared according to WO 2021127166, Acid AG, 50 mg, 0.184 mmol, 1.00 equiv) and 2-[(2R)-1-methylpyrrolidin-2-yl]-1-{[2-(trimethylsilyl)ethoxy]methyl}pyrrolo[3,2-c]pyridin-6-amine (Intermediate 1, 63.63 mg, 0.184 mmol, 1.0 equiv) in Pyridine (2 mL) was added EDCI (70.40 mg, 0.368 mmol, 2.0 equiv) at room temperature. The resulting mixture was stirred for 16 h at room temperature and concentrated under vacuum. This resulted in N-{2-[(2R)-1-methylpyrrolidin-2-yl]-1-{[2-(trimethylsilyl)ethoxy]methyl}pyrrolo[3,2-c]pyridin-6-yl}-4-[1-(oxan-2-yl)pyrazol-4-yl]benzamide (100 mg, crude) as a brown oil.

LC-MS: (ES, m/z): [M+H]=601

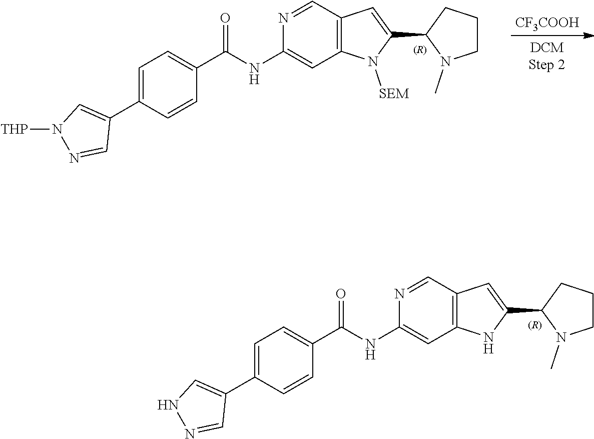

To a stirred solution/mixture of N-{2-[(2R)-1-methylpyrrolidin-2-yl]-1-{[2-(trimethylsilyl)ethoxy]methyl}pyrrolo[3,2-c]pyridin-6-yl}-4-[1-(oxan-2-yl)pyrazol-4-yl]benzamide (100 mg, crude) in DCM was added CF₃COOH (2 mL, 26.926 mmol, 161.78 equiv) at room temperature. The resulting mixture was stirred for 20 h at room temperature. The resulting mixture was concentrated under vacuum. The residue was basified to pH 8 with NH₄₀H (aq.). The crude product was purified by Prep-HPLC with the following conditions: Column, XBridge Shield RP18 OBD Column, 19×150 mm, 5 μm; mobile phase, Water (0.05% NH₃H₂O) and ACN (18% ACN up to 35% in 8 min); Detector, UV 254 nm) to afford N-{2-[(2R)-1-methylpyrrolidin-2-yl]-1H-pyrrolo[3,2-c]pyridin-6-yl}-4-(1H-pyrazol-4-yl)benzamide (30.6 mg, 43.1% for two steps) as a light yellow solid.

LC-MS: (ES, m/z): [M+H]=387

H-NMR: (400 MHz, CD₃OD-d₄, ppm): δ 8.51 (s, 1H), 8.19 (s, 1H), 8.09 (s, 2H), 8.01 (d, J=8.0 Hz, 2H), 7.77 (d, J=8.0 Hz, 2H), 6.51 (s, 1H), 3.45 (t, J=8.0 Hz, 1H), 3.27-3.22 (m, 1H), 2.42 (q, J=8.8 Hz, 1H), 2.31 (s, 3H), 2.32-2.24 (m, 1H), 2.09-1.89 (m, 3H).

### Example 6: (R)-N-(2-(1-methylpiperidin-2-yl)-1H-pyrrolo[3,2-c]pyridin-6-yl)-4-(1H-pyrazol-4-yl)benzamide (Sample 6)

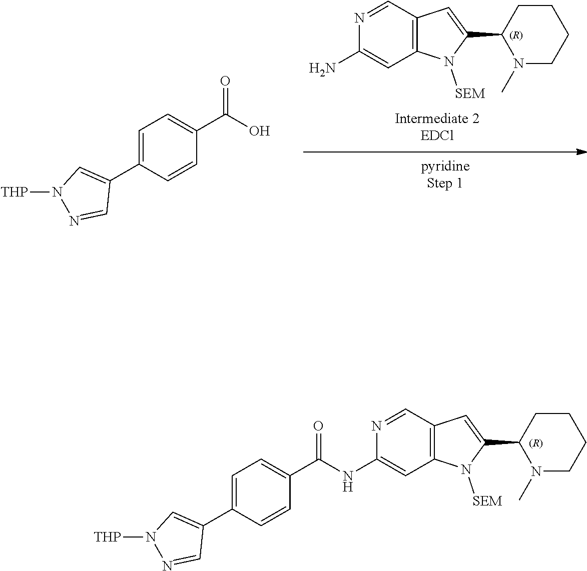

Into a 8 mL vial were added 4-[1-(oxan-2-yl)pyrazol-4-yl]benzoic acid (Prepared according to WO 2021127166, Acid AG, 50 mg, 0.184 mmol, 1 equiv) and EDCI (42.24 mg, 0.221 mmol, 1.2 equiv) in pyridine (5 mL) at room temperature. The resulting mixture was stirred for 10 min. To the above mixture was added 2-[(2R)-1-methylpiperidin-2-yl]-1-{[2-(trimethylsilyl)ethoxy]methyl}pyrrolo[3,2-c]pyridin-6-amine (Intermediate 2, 66.21 mg, 0.184 mmol, 1 equiv). The resulting mixture was stirred for additional 16 h and the crude product was purified by reverse phase flash with the following conditions (column, C18 silica gel; mobile phase, 0.05% NH₃·H₂O in water, MeCN 5% to 60% gradient in 10 min; detector, UV 220 nm)). This resulted in N-{2-[(2R)-1-methylpiperidin-2-yl]-1-{[2-(trimethylsilyl)ethoxy]methyl}pyrrolo[3,2-c]pyridin-6-yl}-4-[1-(oxan-2-yl)pyrazol-4-yl]benzamide (79 mg, 69.97%) as a white solid

LC-MS (ES, m/z): [M+1]⁺=615.3

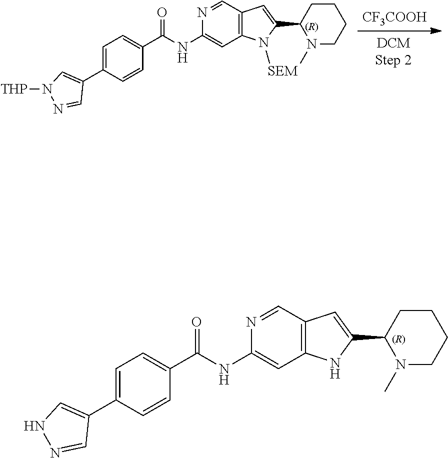

Into a 8 mL vial were added N-{2-[(2R)-1-methylpiperidin-2-yl]-1-{[2-(trimethylsilyl)ethoxy]methyl}pyrrolo[3,2-c]pyridin-6-yl}-4-[1-(oxan-2-yl)pyrazol-4-yl]benzamide (79 mg, 0.128 mmol, 1 equiv) in DCM (5 mL), and trifluoroacetic acid (1 mL) were added at room temperature. The resulting mixture was stirred for additional 1 h at room temperature and concentrated under reduced pressure. The residue was dissolved in DMF (1 mL) and adjusted to pH 10 with NH₃·H₂O. The crude product (50 mg) was purified by Prep-HPLC with the following conditions: (0.1% NH₃·H₂O in water and MeCN (20% upto 60% in 8 min)) to afford N-{2-[(2R)-1-methylpiperidin-2-yl]-1H-pyrrolo[3,2-c]pyridin-6-yl}-4-(1H-pyrazol-4-yl)benzamide (10.3 mg, 20.02%) white solid.

LC-MS (ES, m/z): [M+1]⁺=401

¹H-NMR: ¹H NMR (400 MHz, Methanol-d4) δ 8.55 (s, 1H), 8.21 (s, 1H), 8.17 (m, 2H), 8.04 (d, J=8.4 Hz, 2H), 7.79 (d, J=8.3 Hz, 2H), 6.52 (s, 1H), 3.18 (m, 1H), 3.09 (s, 1H), 2.25-2.18 (s, 1H), 2.12 (s, 3H), 2.03-1.83 (m, 3H), 1.79-1.56 (m, 2H), 1.49-1.38 (m, 1H).

### Example 7: (R)-N-(2-(1-methylpiperidin-2-yl)-1H-pyrrolo[3,2-c]pyridin-6-yl)-5-(1H-pyrazol-4-yl)picolinamide (Sample 7)

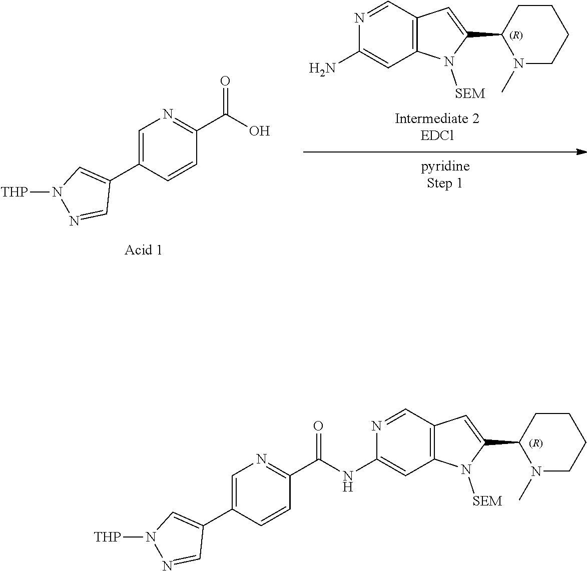

Into a 8 mL vial were added 5-(1-(tetrahydro-2H-pyran-2-yl)-1H-pyrazol-4-yl)picolinic acid (Acid 1, 50 mg, 0.183 mmol, 1 equiv) and EDCI (42.09 mg, 0.220 mmol, 1.2 equiv) in pyridine (1 mL) at room temperature. The resulting mixture was stirred for 10 min at room temperature. To the above mixture was added 2-[(2R)-1-methylpiperidin-2-yl]-1-{[2-(trimethylsilyl)ethoxy]methyl}pyrrolo[3,2-c]pyridin-6-amine (Intermediate 2, 65.97 mg, 0.183 mmol, 1 equiv). The resulting mixture was stirred for additional 16 h at room temperature. The crude product was purified by reverse phase flash with the following conditions (column, C18 silica gel; mobile phase, 0.05% NH₃·H₂O in water, MeCN 5% to 60% gradient in 10 min; detector, UV 220 nm)). This resulted in N-{2-[(2R)-1-methylpiperidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide (100 mg, 88.75%) as a white solid.

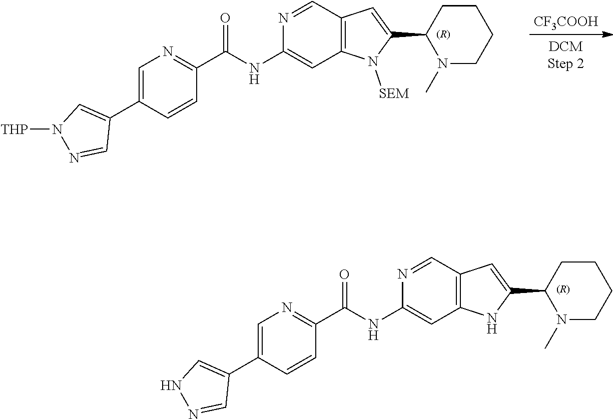

Into a 8 mL vial were added N-{2-[(2R)-1-methylpiperidin-2-yl]-1-{[2-(trimethylsilyl)ethoxy]methyl}pyrrolo[3,2-c]pyridin-6-yl}-5-[1-(oxan-2-yl)pyrazol-4-yl]pyridine-2-carboxamide (100 mg, 0.162 mmol, 1 equiv) in DCM (10 mL) and trifluoroacetic acid (3 mL) at room temperature. The resulting mixture was stirred for additional 1 h at room temperature concentrated under reduced pressure. The residue was diluted with DMF (1 mL), adjusted to pH 10 with NH₃·H₂O and the crude product (50 mg) was purified by Prep-HPLC with the following conditions (0.1% NH₃·H₂O in water and MeCN (20% upto 60% in 8 min)) to afford N-{2-[(2R)-1-methylpiperidin-2-yl]-1H-pyrrolo[3,2-c]pyridin-6-yl}-5-(1H-pyrazol-4-yl)pyridine-2-carboxamide (14.3 mg, 21.94%) white solid.

LC-MS (ES, m/z): [M+1]⁺=402.2

¹H-NMR ¹H NMR (400 MHz, Methanol-d4) δ 9.02 (s, 1H), 8.54 (s, 1H), 8.30 (s, 1H), 8.21-8.16 (m, 3H), 8.10 (m, 1H), 6.57 (s, 1H), 3.18 (m, 1H), 3.03 (m, 1H), 2.37 (m, 1H), 2.23-2.18 (s, 3H), 1.96-1.92 (m, 3H), 1.72-1.84 (m, 2H), 1.60-1.53 (m, 1H).

### Example 8: (R)-4-(5-(methoxymethyl)-1H-pyrazol-4-yl)-N-(2-(1-methylpyrrolidin-2-yl)-1H-pyrrolo[3,2-c]pyridin-6-yl)benzamide (Sample 8)

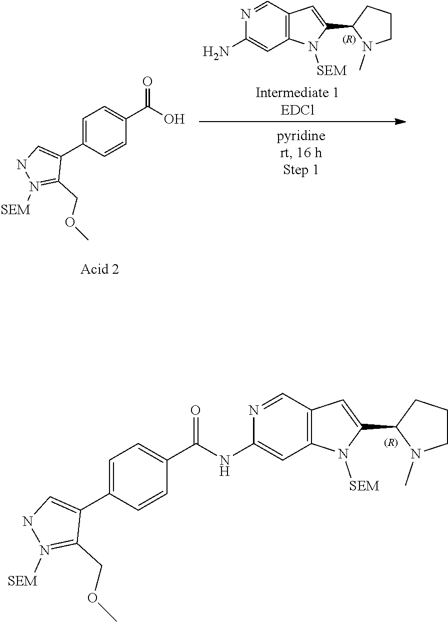

A mixture of 4-[5-(methoxymethyl)-1-{[2-(trimethylsilyl)ethoxy]methyl}pyrazol-4-yl]benzoic acid (Acid 2, 50 mg, 0.138 mmol, 1 equiv) and 2-[(2R)-1-methylpyrrolidin-2-yl]-1-{[2-(trimethylsilyl)ethoxy]methyl}pyrrolo[3,2-c]pyridin-6-amine (Intermediate 1, 75 mg, 0.216 mmol, 1.57 equiv), and EDCI (50 mg, 0.261 mmol, 1.89 equiv) in pyridine (3 mL, 0.038 mmol, 0.27 equiv) was stirred for 16 h at room temperature. The reaction mixture was concentrated under vacuum to afford 4-[5-(methoxymethyl)-1-{[2-(trimethylsilyl)ethoxy]methyl}pyrazol-4-yl]-N-{2-[(2R)-1-methylpyrrolidin-2-yl]-1-{[2-(trimethylsilyl)ethoxy]methyl}pyrrolo[3,2-c]pyridin-6-yl}benzamide (60 mg) which was used directly in the next step.

LC-MS (ES, m/z): [M+1]⁺=691.3

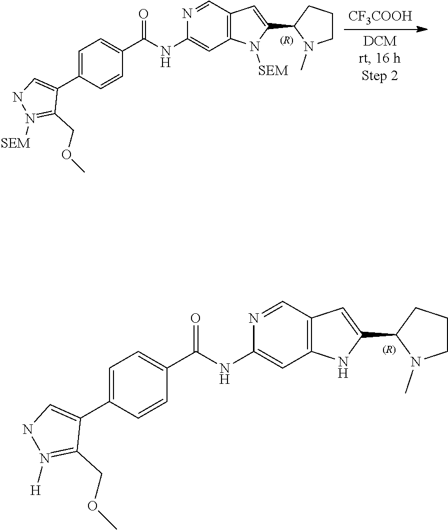

Into a solution of DCM (6 mL) and 4-[5-(methoxymethyl)-1-{[2-(trimethylsilyl)ethoxy]methyl}pyrazol-4-yl]-N-{2-[(2R)-1-methylpyrrolidin-2-yl]-1-{[2-(trimethylsilyl)ethoxy]methyl}pyrrolo[3,2-c]pyridin-6-yl}benzamide (60 mg, 0.087 mmol) was added trifluoroacetic acid (2 mL, 0.009 mmol, 0.10 equiv) at RT. The resulting mixture was stirred for 1 h and concentrated under reduced pressure. The crude product was purified by Prep-HPLC with the following conditions: Column, SunFire Prep C18 OBD Column, 19×150 mm 5 m 10 nm; mobile phase, Water (0.1% NH₃H₂O) and ACN (28% ACN up to 63% in 9 min); Total flow rate, 20 mL/min; Detector, UV 220 nm. This resulted in (R)-4-(5-(methoxymethyl)-1H-pyrazol-4-yl)-N-(2-(1-methylpyrrolidin-2-yl)-1H-pyrrolo[3,2-c]pyridin-6-yl)benzamide (28.6 mg, 75.1%) as a white solid.

LC-MS (ES, m/z): [M+1]⁺=431.3

1H NMR (400 MHz, DMSO-d6) δ 8.50 (d, J=1.1 Hz, 1H), 8.18 (s, 1H), 8.02 (m, 3H), 7.72 (m, 2H), 6.50 (s, 1H), 4.58 (s, 2H), 3.41 (m, 4H), 3.21 (t, J=7.5 Hz, 1H), 2.38 (m, 1H), 2.28 (m, 4H), 2.02 (m, 2H), 1.95-1.86 (m, 1H).

### Example 9: (R)-4-(5-fluoro-1H-pyrazol-4-yl)-N-(2-(1-methylpyrrolidin-2-yl)-1H-pyrrolo[3,2-c]pyridin-6-yl)benzamide (Sample 9)

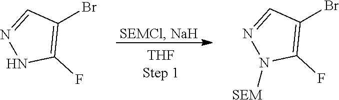 [SMILES: Fc1[nH]ncc1Br ; C[Si](C)(C)CCOCn1ncc(Br)c1F]

Into a 40-mL vial, was placed 4-bromo-3-fluoro-2H-pyrazole (100 mg, 0.606 mmol, 1 equiv), [2-(chloromethoxy)ethyl]trimethylsilane (151.60 mg, 0.909 mmol, 1.5 equiv), NaH (29.09 mg, 1.212 mmol, 2.0 equiv) and THE (1 mL). The resulting solution was stirred for 4 h at 25° C. The reaction was monitored by LCMS. The residue was purified by reverse flash chromatography with the following conditions: column, C18 silica gel; mobile phase, Water (0.1% FA) and ACN (5.0% ACN up to 45.0% in 7 min); Total flow rate, 20 mL/min; Detector, UV 220 nm. The collected fractions were combined and concentrated under vacuum. This resulted in 4-bromo-5-fluoro-1-{[2-(trimethylsilyl)ethoxy]methyl}pyrazole (110 mg, 37.49%) as a light yellow oil.

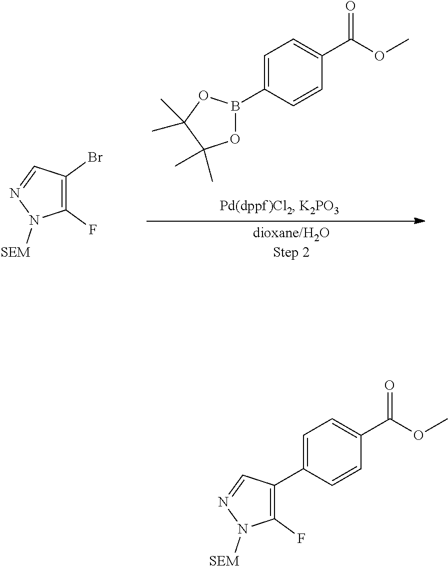

Into a 8-ml vial, was placed 4-bromo-5-fluoro-1-{[2-(trimethylsilyl)ethoxy]methyl}pyrazole (100 mg, 0.339 mmol, 1 equiv), methyl 4-(4,4,5,5-tetramethyl-1,3,2-dioxaborolan-2-yl)benzoate (115.42 mg, 0.441 mmol, 1.3 equiv), Pd(dppf)Cl₂ (12.39 mg, 0.017 mmol, 0.05 equiv), K₂CO₃ (117.03 mg, 0.848 mmol, 2.5 equiv), and dioxane (1 mL) and H₂O (0.1 mL). The resulting solution was stirred for 16 h at 105° C. The reaction was cooled and diluted with water and extracted with EA. The organic layer was washed with brine, dried with Na₂SO₄ and concentrated under vacuum. The residue was applied on a silica gel column and eluted with ethyl acetate/hexane (1/5). The collected fractions were combined and concentrated under vacuum. This resulted in methyl 4-(5-fluoro-1-{[(trimethylsilyl)methoxy]methyl}pyrazol-4-yl)benzoate (71 mg, 60.2%) as a off-white solid.

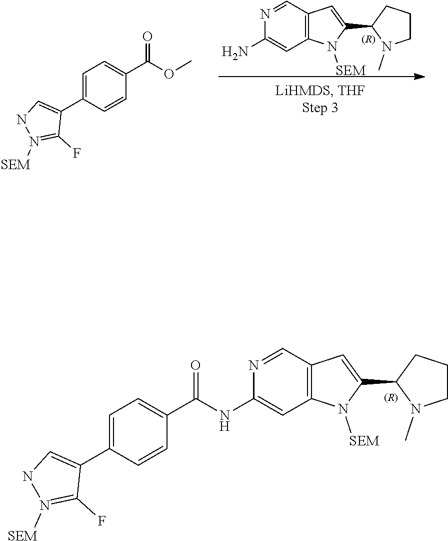

Into a 8 ml vial, methyl 4-(5-fluoro-1-{[2-(trimethylsilyl)ethoxy]methyl}pyrazol-4-yl)benzoate (70 mg, 0.200 mmol, 1 equiv), 2-[(2R)-1-methylpyrrolidin-2-yl]-1-{[2-(trimethylsilyl)ethoxy]methyl}pyrrolo[3,2-c]pyridin-6-amine (Intermediate 1, 69.22 mg, 0.200 mmol, 1.0 equiv) and THE (1 mL) were added and stirred. LiHMDS (200.53 mg, 1.200 mmol, 6.0 equiv) was added dropwise in an ice water bath, and then stirred for 5 min. The reaction was warmed to room temperature and stirred for 2 h. The reaction was then quenched by the addition of 50 mL of NH₄Cl (aq.) and extracted with EA. The resulting mixture was washed with brine, dried over sodium sulfate, and concentrated under reduced pressure. This resulted in 4-(5-fluoro-1-{[2-(trimethylsilyl)ethoxy]methyl}pyrazol-4-yl)-N-{2-[(2R)-1-methylpyrrolidin-2-yl]-1-{[2-(trimethylsilyl)ethoxy]methyl}pyrrolo[3,2-c]pyridin-6-yl}benzamide (90 mg, 67.8%) as a brown oil.

LC-MS (ES, m/z): [M+1]⁺=665

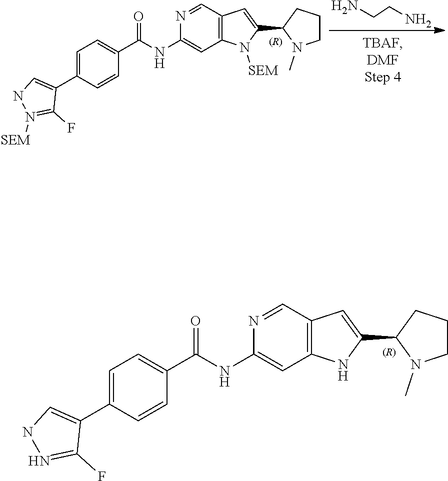

In an 8 ml vial, was placed 4-(5-fluoro-1-{[2-(trimethylsilyl)ethoxy]methyl}pyrazol-4-yl)-N-{2-[(2R)-1-methylpyrrolidin-2-yl]-1-{[2-(trimethylsilyl)ethoxy]methyl}pyrrolo[3,2-c]pyridin-6-yl}benzamide (70 mg, 0.105 mmol, 1 equiv), ethylenediamine (126.53 mg, 2.100 mmol, 20.0 equiv), TBAF (412.85 mg, 1.575 mmol, 15.0 equiv), and DMF (1 mL). The resulting solution was stirred for 8 h at 70° C. The reaction mixture was concentrated under vacuum. The crude product was purified by Prep-HPLC with the following conditions (Prep-HPLC-013): Column, SunFire Prep C18 OBD Column, 19×150 mm 5 m; mobile phase, Water (0.1% FA) and ACN (5.0% ACN up to 45.0% in 7 min); Total flow rate, 20 mL/min; Detector, UV 220 nm. Lyophilization resulted in 4-(3-fluoro-2H-pyrazol-4-yl)-N-{2-[(2R)-1-methylpyrrolidin-2-yl]-1H-pyrrolo[3,2-c]pyridin-6-yl}benzamide (18.5 mg, 42.5%) as a white solid.

LC-MS (ES, m/z): [M+1]⁺=405

H-NMR (400 MHz, DMSO-d6) δ 12.73 (s, 1H), 11.38 (s, 1H), 10.44 (s, 1H), 8.51 (s, 1H), 8.33 (t, J=2.0 Hz, 1H), 8.21 (d, J=1.0 Hz, 1H), 8.14-8.07 (m, 2H), 7.73-7.67 (m, 2H), 6.40 (s, 1H), 3.29 (s, 1H), 3.15 (t, J=8.0 Hz, 1H), 2.36-2.22 (m, 1H), 2.18 (s, 4H), 1.93-1.80 (m, 3H).

### Example 10

### FRET Assay:

Compounds of the invention were tested in a TR-FRET ENL Screening Assay. TR-FRET (time-resolved fluorescence energy transfer) can be used to quantify ENL YEATS domain binding to a crotonylated histone peptide (H3K9cr, aa1-20). Streptavidin-Europium (Eu) chelate binds the biotinylated peptide, while Anti-6×HIS ULight™ binds 6×HIS-ENL. When Eu chelate is excited at 320 nm, fluorescence resonance energy transfer (FRET) occurs if Eu and ULight are made proximal by ENL binding to the acyl-peptide. ULight emission (FRET) is measured at 665 nm and normalized to the Eu emission at 615 nm to reduce variability between wells.

### FRET Assay Protocol

Compounds of the invention were dissolved in DMSO at a concentration of 3 mM with subsequent dilutions in assay buffer (50 mM HEPES PH 7.0, 150 mM NaCl, 0.05% BSA, 0.2% Pluronic F-127) such that the assay contained 1% DMSO. In a white 384 shallow well Microplate (Proxiplate-384 Plus, PerkinElmer, 6008280), 150 nL of compound or vehicle (1% DMSO in assay buffer) for the high control (HC) wells and 5 μL of 30 nM ENL Protein (6×HIS ENL YEATS Domain, EpiCypher, 15-0069) were combined and incubated 15 minutes at RT. Low control (LC) wells received 5 μL of assay buffer instead of ENL protein. Then 5 μL of 15 nM H3K9cr peptide (H3 aa1-20, biotinylated; EpiCypher, 12-0099) in assay buffer was added and incubated 30 minutes at RT. Finally, a 5 L mix of 45 nM Anti-6HIS ULight (PerkinElmer, TRF0105) and 1.5 nM Streptavidin-Europium Chelate (PerkinElmer, AD0060) were added and incubated for a further 30 minutes at RT. The TR-FRET signal (665 nm signal/615 nm signal×10,000) was measured using a PerkinElmer 2104 EnVision (Xenon Flash Lamp excitation, 320 nm±37.5 nm excitation filter, 407 nm cut off dichroic mirror, 615 nm±4.25 (Europium) nm and 665 nm+3.75 nM (ULight) emission filters). Compound concentration response curves were performed in duplicate over the concentration range of 0.15 nM-30 μM. The response at each compound concentration minus the LC value was converted to percent inhibition of the vehicle control group response (HC-LC). The relationship between the % inhibition and the compound concentration was analyzed using a four parameter logistic equation to estimate lower and upper asymptotes, the compound concentration producing 50% inhibition (IC50 value) and the slope at the mid-point location.

TABLE 1
FRET Assay Results

| Example/Sample | TR FRET ENL IC50 (μM) |
|---|---|
| 1 | >30 |
| 2 | 0.195 |
| 3 | >13 |
| 4 | 0.067 |
| 5 | 0.046 |
| 6 | 0.064 |
| 7 | 0.39 |
| 8 | 0.179 |
| 9 | 0.073 |

### Example 11: Cell Assay

Cell-based assays were used to assess the ability of test compounds to reduce cell viability in both MV4:11 (MLL-AF4 MLL) and K562 cells, which were cultured in Iscove's Modified Dulbecco's medium (Gibco, 12440061) containing 10% FBS. The assays were conducted over 12 days and the cells being split on days 4 and 8. Compound concentration response curves were performed in duplicate over the concentration range of 0.15 nM-30 μM. On day 0, the compounds or vehicle were plated in a 300 nL directly into 96 well cell culture plates (Corning, 3599) with 5000 cells/well in a volume of 100 μL. Blank wells received cell culture medium. Plates were incubated for 4 days at 37° C. with 5% CO₂. On days 4 and day 8 the cells were split and incubated for a further 4 days whilst an aliquot of cells were taken for the CTG readout. For the cell splitting, 270 nL of compounds or DMSO was added to a new 96 well cell culture plate to which 90 μL of medium plus 10 μL of cells from the original assay plate (after mixing) or 100 μL of medium (Blank wells) was added. This was repeated on day 8.

Cell viability was assessed using the CellTiter-Glo® homogeneous luminescent assay kit (Promega, G9243), according to the manufacturer's instructions. This quantifies ATP, which indicates the presence of metabolically active cells. On days 4, 8 and 12, 20 μl of the remaining cell suspension was aspirated into 384-well plate (Corning 3570) to which an equal volume CellTiter-Glo reagent was. Plates were incubated for 10 minute incubation at RT prior to recording the luminescence signal using EnVision plate reader (PE, 2104). The resulting data were analyzed as follows:

$$Inhibition ( \% ) = 100 \% \times ( {Lum}_{vehicle} - {Lum}_{sample} ) / ( {Lum}_{vehicle} - {Lum}_{blank} )$$

where vehicle are cells treated with 0.3% DMSO, Blank is culture medium. IC₅₀ determinations were calculated by fitting the curve using XLfit (v5.3.1.3): Y=Bottom+(Top−Bottom)/(1+10\hat{ }((Log IC50−X)*HillSlope)).

TABLE 2
Cell Assay Results

| Example/Sample | MV4:11; 12 Day IC₅₀ |
|---|---|
| 1 | 0.040 |
| 2 | 0.019 |
| 3 | 0.083 |
| 4 | 0.119 |
| 5 | 0.079 |
| 6 | 0.138 |
| 7 | 0.036 |
| 8 | 0.357 |
| 9 | 0.176 |

### Example 12: FLT3 Activity

### FLT3 Inhibition Assay

| Reagent | Vendor | Cat No. |
|---|---|---|
| FLT3 | Carna | 08-154 |
| Staurosporine | BioAustralis | BIA-S1086 |
| FL Peptide 2 | PerkinElmer | 760346 |

Solution 1

| Component | Concentration |
|---|---|
| MgCl₂ | 10 mM |
| Brij-35 | 0.01% |
| DTT | 2 mM |
| BSA | 0.05% |
| EGTA | 1 mM |
| HEPE (pH 7.5) | 50 mM |

Starting with a 10 mM stock solution, each test compound was serially diluted into 10 concentrations by 3-fold dilution using TECAN EVO200. 60 nL of each stock was transferred to a 384 plate using Echo550.

Solution 1 was prepared as the table above and used to dilute the FLT3, ATP and FL2 stock to 0.9379 nM (1.33×), 400 μM (4×) and 6 μM (4×) respectively. The FLT3 solution (15 μL) at 25° C. was added to each well, shaken for 1 min and preincubated with test compound and controls for 30 min. To each well, 5 μL of ATP and FL-Peptide2 solutions, as prepared above, were added and the plate was shaken for 10 seconds, then spun briefly at 1000 rpm, then incubated for 90 minutes at room temperature. The plate was read on Caliper EZ reader, and the IC₅₀ values were calculated using XLfit (equation below).

$$\% inhibition = 100 \% \times ( {Lum}_{HC} - {Lum}_{Sample} ) / ( {Lum}_{HC} - {Lum}_{LC} )$$

Lum_{Sample}: Test compound signal
Lum_{LC}: Low control signal
Lum_{HC}: High control signal

TABLE 3
FLT3 Assay Results

| Example/Sample | FLT3 IC₅₀ (μM) |
|---|---|
| 1 | 0.007 |
| 2 | 0.007 |
| 3 | 0.013 |
| 4 | ND† |
| 5 | 0.274 |
| 6 | ND |
| 7 | ND |
| 8 | ND |
| 9 | ND |

†not determined

1. A compound of Formula I: 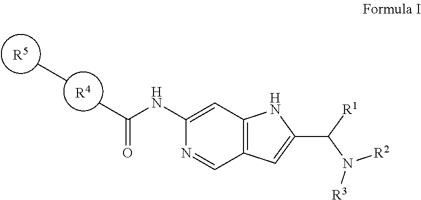
wherein:
R¹ and R² taken together form a pyrrolidine or piperidine;
R³ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle;
C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

2. A compound of claim 1, wherein the compound belongs to Formula Ia′: 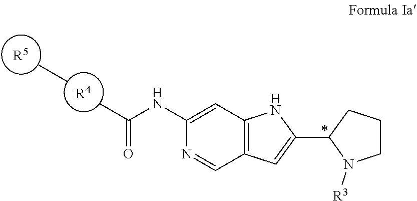
wherein R³, R⁴ and R⁵ are as defined above for Formula I.

3. A compound of claim 1, wherein the compound belongs to Formula Ia″: 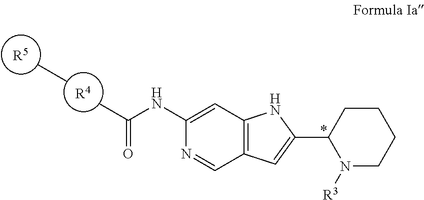
wherein R³, R⁴ and R⁵ are as defined above for Formula I.

4. A compound of claim 2 or 3, wherein:
R⁴ is selected from the group consisting of benzene, pyridine, pyrimidine, pyridazine and pyrazine, optionally substituted with a R⁷ group as defined above for Formula I; and
R⁵ is selected from the group consisting of pyrrolidine; pyrroline; pyrazolidine; pyrazoline; imidazoline; imidazoline; pyrrole; pyrazole; imidazole; triazole; isoxazole; oxazole; 1,2,3-oxadiazole; 1,3,4-oxadiozole; furazan; 1,2,4-oxadiazole; 1,2,3,4-oxatriazole; 1,2,3,5-oxatriazole; isothiazole; thiazole; 1,2,3-thiadiazole; 1,3,4-thiadizaole; 1,2,5-thadiazole; 1,2,4-thiadiazole; 1,2,3,4-thiatriazole; 1,2,3,5-thiatriazole; furan and thiophene, optionally substituted with a R⁶ group as defined above for Formula I.

5. A compound of any one of claim 2, 3, or 4, wherein R³ is methyl.

6. A compound of Formula II: 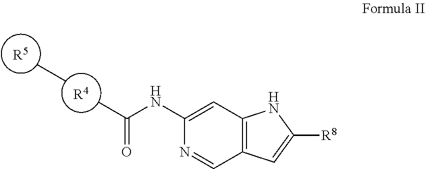
wherein:
R⁸ is selected from hydrogen and C₁-C₈ alkyl;
R⁴ is an aromatic 5- or 6-membered carbocycle or heterocycle optionally substituted with one or more R⁷ groups selected from C₁-C₈ alkyl; C₁-C₁₀ haloalkyl; C₃-C₈ carbocycle;
C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen; and
R⁵ is a 5- or 6-membered carbocycle or heterocycle optionally substituted with one or more R⁶ groups selected from C₁-C₈ alkyl; C₁-C₁₀ haloalkyl; C₃-C₈ carbocycle; C₁-C₁₀ oxaalkyl, —SO₂(C₁₋₆)alkyl; —SO₂NH(C₀₋₃H₁₋₇); —CONH(C₀₋₃H₁₋₇); —SO₂NH(C₁₋₆)oxaalkyl; —CN; —CH₂CN; —CH₂NH₂; —NH₂, —NR¹⁴, where R¹⁴ is independently chosen from hydrogen, (C₁₋₆)fluoroalkyl, and (C₁₋₃)oxaalkyl, —CH₂OH, benzyloxy, —C(═NH)—NH₂; oxo; and halogen.

7. A compound of claim 6, wherein
R⁴ is selected from the group consisting of benzene, pyridine, pyrimidine, pyridazine and pyrazine, optionally substituted with a R⁷ group as defined above for Formula I; and
R⁵ is selected from the group consisting of pyrrolidine; pyrroline; pyrazolidine; pyrazoline; imidazoline; imidazoline; pyrrole; pyrazole; imidazole; triazole; isoxazole; oxazole; 1,2,3-oxadiazole; 1,3,4-oxadiozole; furazan; 1,2,4-oxadiazole; 1,2,3,4-oxatriazole; 1,2,3,5-oxatriazole; isothiazole; thiazole; 1,2,3-thiadiazole; 1,3,4-thiadizaole; 1,2,5-thadiazole; 1,2,4-thiadiazole; 1,2,3,4-thiatriazole; 1,2,3,5-thiatriazole; furan and thiophene, optionally substituted with a R⁶ group as defined above for Formula I.

8. A compound of claim 6 or 7, wherein R⁸ is methyl.

9. A compound of claim 1 or claim 6, selected from the following group:

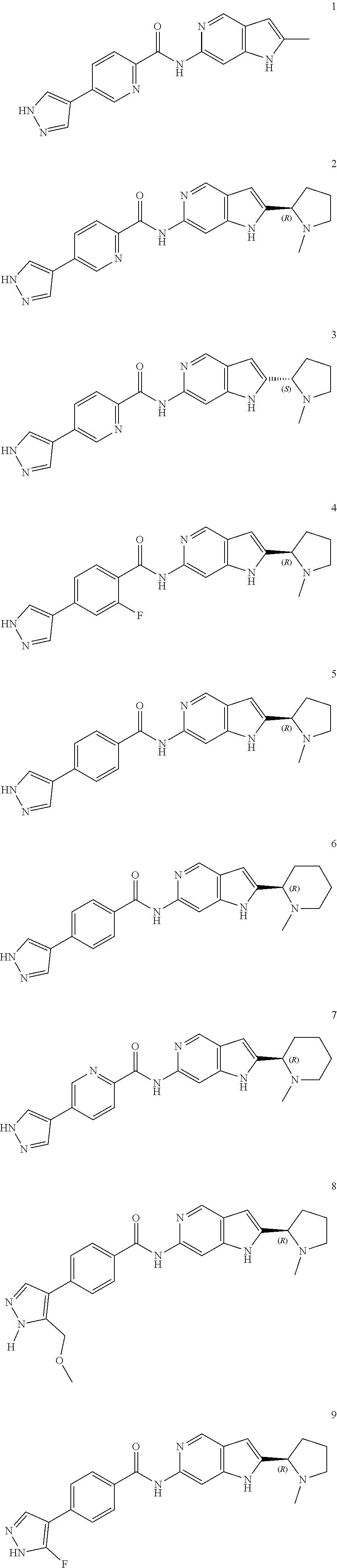

10. A pharmaceutical composition comprising a compound of any of claims 1-9 and one or more pharmaceutically acceptable carriers.

11. The pharmaceutical composition of claim 10, further comprising one or more therapeutic agents.

12. The pharmaceutical composition of claim 11, wherein the one or more therapeutic agent is selected from the group consisting of Bcl-2 inhibitors, cyclin-dependent kinase 4 and 6 (CDK 4/6 inhibitors), DNA methyltransferase inhibitors, histone deacetylase (HDAC) inhibitors, mTOR inhibitors, mutant isocitrate dehydrogenase (IDH1 and IDH2) inhibitors, glucocorticoids, an epigenetic modulators and chemotherapeutic agents.

13. A method of treating an acute leukemia comprising administering a therapeutically effective amount of a compound of any of claims 1-9 or a pharmaceutical composition of claims 10-12 to a subject in need thereof.

14. The method of claim 13, wherein the acute leukemia is acute lymphoblastic leukemia (ALL).

15. The method of claim 13, wherein the acute leukemia is acute myelogenous leukemia (AML).

16. The method of claim 15, wherein the AML is a subtype selected from the group consisting of acute myeloid leukemia, minimally differentiated (MO), acute myeloid leukemia without maturation (M1), acute myeloid leukemia with maturation (M2), acute myeloid leukemia with maturation with t(8;21), acute promyelocytic leukemia (M3), hypergranular type, microgranular type, acute myelomonocytic leukemia (M4), acute myelomonocytic leukemia with increased marrow eosinophils (M4E0), acute monocytic leukemia (M5), acute monoblastic leukemia (M5a), acute monocytic leukemia with maturation (M5b), erythroleukemia erythroid/myeloid (M6a), pure erythroid malignancy (M6b), acute megakaryoblastic leukemia (M7), acute megakaryoblastic leukemia associated with t(1;22), acute basophilic leukemia, acute myelofibrosis (acute myelodysplasia with myelofibrosis), acute leukemia and transient myeloproliferative disorder in Down's Syndrome, hypocellular acute myeloid leukemia, and myeloid sarcoma.

17. The method of claim 13, wherein the at least one compound is administered orally.

18. The method of claim 13, wherein the at least one compound is administered from one to four times per day.
